(19) United States
(12) Patent Application Publication
(10) Pub. No.: US 2014/0107333 A1
(43) Pub. Date: Apr. 17, 2014

(54) POLYHEDRAL CAGE-CONTAINING METALLOPORPHYRIN FRAMEWORKS, METHODS OF MAKING, AND METHODS OF USING

(71) Applicants: Shengqian Ma, Tampa, FL (US); Peter X. Zhang, Tampa, FL (US); Xisen Wang, Tampa, FL (US); Le Meng, Tampa, FL (US); Qigan Cheng, Tampa, FL (US)

(72) Inventors: Shengqian Ma, Tampa, FL (US); Peter X. Zhang, Tampa, FL (US); Xisen Wang, Tampa, FL (US); Le Meng, Tampa, FL (US); Qigan Cheng, Tampa, FL (US)

(21) Appl. No.: 14/125,480

(22) PCT Filed: Jun. 15, 2012
(86) PCT No.: PCT/US12/42670

§ 371 Date: Dec. 11, 2013

Related U.S. Application Data
(60) Provisional application No. 61/497,816, filed on Jun. 16, 2011.

(51) Int. Cl. C07D 487/22

(52) U.S. Cl. CPC C07D 487/22
USPC 540/145

(57) ABSTRACT

Embodiments of the present disclosure provide compositions including metal-organic polyhedrons, metalloporphyrin framework structures, methods of making these, methods of using these, and the like.

### CROSS-REFERENCE TO RELATED APPLICATION

This application claims priority to U.S. provisional application entitled “POLYHEDRAL CAGE-CONTAINING METALLOPORPHYRIN FRAMEWORKS,” having Ser. No. 61/497,816, filed on Jun. 16, 2011, which is entirely incorporated herein by reference.

### BACKGROUND

Metal-organic framework (MOF) materials have received extensive interest due to their potential applications for gas storage, sensors, and particularly heterogeneous catalysis. However, many of the currently used MOFs have limitations and thus, other types of MOFs are needed to achieve these desired properties.

### SUMMARY

Embodiments of the present disclosure provide compositions including metal-organic polyhedrons, metalloporphyrin framework structures, methods of making these, methods of using these, and the like.

An embodiment of the composition, among others, includes: a metal-metalloporphyrin framework that includes a porphyrin ligand and a secondary building unit, wherein the porphyrin ligand is represented by formula A:

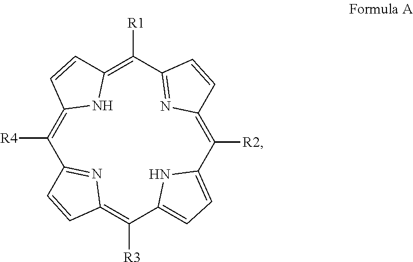

wherein one or more of the R1, R2, R3, and R4, includes a functional group that bonds with the secondary building unit, wherein R1, R2, R3, and R4 are independently selected from H and a moiety having one of more functional groups selected from the group consisting of: —CO₂H, —CS₂H, —NO₂, —B(OH)₂, —SO₃H, —CN, -tetrazolate, -1,2,3 or 1,2,4-triazolate, -pyrazolate, —PO₃H, and -pyridyl; wherein at least one of R1, R2, R3, and R4 is not H.

An embodiment of the metal-organic polyhedron (MOP), among others, includes: a porphyrin ligand and a secondary building unit, wherein the one or more of the R1, R2, R3, and R4, include a functional group that bonds with the secondary building unit; wherein the porphyrin ligand is represented by formula A:

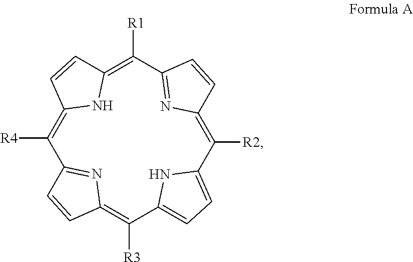

wherein R1, R2, R3, and R4 are independently selected from H and a moiety having one of more functional groups selected from the group consisting of: —CO₂H, —CS₂H, —NO₂, —B(OH)₂, —CN, -tetrazolate, -1,2,3 or 1,2,4-triazolate, -pyrazolate, —PO₃H, and -pyridyl; wherein at least one of R1, R2, R3, and R4 is not H.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIG. 1.1(a) illustrates a nanoscopic cage enclosed by eight dicopper paddlewheel SBUs and sixteen bdcpp ligands (eight are face-on porphyrins and the other eight only provide isophthalate units). FIG. 1.1(b) illustrates one layer of nanoscopic cages extended in the ab plane (hydrogen atoms omitted for clarity).

FIG. 1.2(a) is an illustration of linking bdcpp ligand and dicopper paddlewheel to form the irregular rhombicuboctahedral cage. FIG. 1.2(b) illustrates “ABAB” packing of rhombicuboctahedron layers in MMPF-1. FIG. 1.2(c) illustrates space filling model on the [1 0 0] plane indicating the open pore size of ˜3.3”3.4 A.

FIG. 1.3 illustrates gas adsorption isotherms of MMPF-1: FIG. 1.3(a) 77K; FIG. 1.3(b) 195 K.

FIG. 1.4 illustrates scheme 1. FIG. 1.4(a) illustrates 5,15-bis(3,5-dicarboxyphenyl)porphyrin (bdcpp) ligand and FIG. 1.4(b) illustrates dicopper paddlewheel SBU.

FIG. 1.5 illustrates three types of windows in the porphyrin cage of MMPF-1: (a) square; (b) rectangular; (c) triangular (hydrogen atoms omitted for clarity).

FIG. 1.6 illustrates lvt topology of MMPF-1.

FIG. 1.7 illustrates small apertures observed in MMPF-1 along (a) [0 1 0] direction; (b) [1 1 1] direction.

FIG. 1.8 illustrates TGA plot of MMPF-1.

FIG. 1.9 illustrates CO₂ adsorption isotherm at 195 K for MMPF-1 activated at 200° C.

FIG. 1.10 illustrates powder X-ray patterns of MMPF-1.

FIG. 1.11 illustrates Table S1: crystal data and structure refinement for MMPF-1.

FIG. 2.1(a) illustrates three cobalt porphyrins located in the “face-to-face” configuration in MMPF-2. FIG. 2.1(b) illustrates space filling model of three types of channels in MMPF-2 viewed from the c direction.

FIG. 2.2 illustrates Ar adsorption isotherm of MMPF-2 at 87 K (insert DFT pore size distribution).

FIG. 2.3(a) illustrates CO₂ and N₂ adsorption isotherms of MMPF-2 at 273 K and 298 K, while FIG. 2.3(b) illustrates isosteric heats of adsorption of MMPF-2 for CO₂.

FIG. 2.4 illustrates embodiments of the present disclosure.

FIG. 2.5 illustrates TGA plot of MMPF-2.

FIG. 2.6 illustrates msq topology of MMPF-2.

FIG. 2.7 illustrates powder X-Ray patterns of MMPF-2.

FIG. 2.8 illustrates N₂ adsorption isotherm of MMPF-2 at 77K (Langmuir surface area (P/P₀=0.9): 2005 m²/g; BET surface area (P/P₀=0.02-0.2): 1420 m²/g).

FIG. 2.9 illustrates O₂ adsorption isotherm of MMPF-2 at 87K (Langmuir surface area (P/P₀=0.9): 2041 m²/g; BET surface area (P/P₀=0.02˜0.2): 1406 m²/g).

FIG. 2.10 illustrates nonlinear curve fitting of CO₂ adsorption isotherms for MMPF-2 at two 273 K and 298 K.

FIG. 2.11 illustrates coordination and atom numbering scheme for MMPF-2. Atomic displacement ellipsoids are drawn at 50% probability level

FIG. 2.12 illustrates Table S1: list of porphyrin-based MOFs with surface area derived from gas sorption measurements.

FIG. 2.13 illustrates Table S2: crystal data and structure refinement for MMPF-2

FIG. 3.1 illustrates an embodiment of a compound of the present disclosure.

### DISCUSSION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method may be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

### Discussion:

Embodiments of the present disclosure provide compositions including metal-organic polyhedrons, metalloporphyrin framework structures, methods of making these, methods of using these, and the like.

Embodiments of the present disclosure provide for metalloporphyrin-based nanoscopic polyhedral cages, where the cage walls are rich in π-electron density can provide favorable interactions with targeted substrates. These cages may also contain multiple active metal centers that could facilitate synergistic interactions with substrates. In addition, embodiments of the present disclosure can be used in applications such as gas storage, sensors, and particularly heterogeneous catalysis. For example, metalloporphyrin nanoscopic polyhedral cages can be built into MOFs so that the π-electron rich cage walls together with the high density of open metal sites within the confined nanospace would be conducive to gas storage and/or catalytic performances. Additional details are described in the Examples.

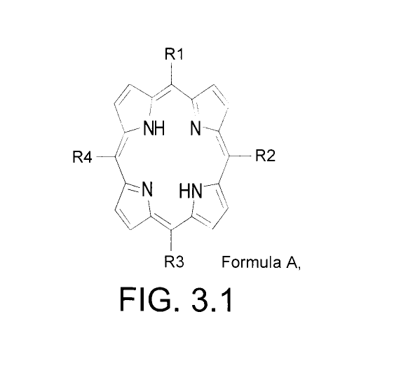

In an embodiment, a metal-organic polyhedron (MOP) can be formed from a porphyrin ligand (See FIG. 3.1) and a secondary building unit (SBU). In an embodiment, the MOP can serve as a supermolecular building block (SBB) that sustains a multidimensional porous metalloporphyrin framework structure exhibiting a very high density of open metal sites in the confined nanoscopic polyhedral cage.

Metal-organic frameworks (MOFs) are materials in which metal to organic ligand interactions can form a porous coordination network. Metal-organic frameworks are coordination polymers with an inorganic-organic hybrid frame comprising metal ions or clusters of metal ions and organic ligands coordinated with the metal ions and/or clusters. These materials are organized in a one-, two- or three-dimensional framework in which the metal clusters are linked to one another periodically by bridging ligands and/or pillar ligands.

In an embodiment, the inorganic sections can be referred to as secondary building units (SBU) and these can include the metal or metal clusters and one or more bridging ligands. SBUs can be connected by pillar ligands (and/or hybrid pillar/bridging ligands) to form the MOPs, which can be used to form MOFs. Typically these materials have a crystal structure. In an embodiment, the polyhedral mesoporous MOF can be stable in water.

In an embodiment, the mesoporous MOF can have a pore size of about 2 nm to 50 nm. In an embodiment, the nanoscopic cage of the mesoporous MOF can have a diameter of about 1 nm to 50 nm. In an embodiment, the mesoporous MOF can have a surface area of about 500 m²/g to 12,000 m²/g.

In an embodiment, the SBU can include units that can bond with a porphyrin ligand of the present disclosure. In particular, the SBU can include a metal and a bridging ligand that can include functional groups that bond with the metals.

As mentioned above, the SBU can include one or more metals. The term “metal” as used within the scope of the present disclosure can refer to metal, metal ions, and/or clusters of metal or metal ions, that are able to form a metal-organic, porous framework material. In an embodiment, the metal can include metals corresponding to the Ia, IIa, IIIa, IVa to VIIIa and Ib and VIb groups of the periodic table of the elements. In an embodiment, the metal (or metal ion) can include: Mg, Ca, Sr, Ba, Sc, Y, Ti, Zr, Hf, V, Nb, Ta, Cr, Mo, W, Mn, Re, Fe, Ru, Os, Co, Rh, Ir, Ni, Pd, Pt, Cu, Ag, Au, Zn, Cd, Hg, Al, Ga, In, Tl, Si, Ge, Sn, Pb, As, Sb and Bi. In an embodiment, the metal ion can have a 1+, 2+, 3+, 4+, 5+, 6+, 7+, or 8+charge.

In an embodiment, the SBU can be selected from the following: a dicopper paddlewheel secondary building unit, a distorted dicobalt trigonal prism secondary building unit. In an embodiment, the SBU can be selected from the following: dimetal (e.g., Mg, Cu, Co, Zn, Mn, Ni, Fe, or Ln metals) square paddlewheel, dimetal (e.g., Mg, Cu, Co, Zn, Mn, Fe, Ni, or Ln metals) triangular paddlewheel, tetra-metal (e.g., Mg, Cu, Co, Zn, Mn, Fe, Ni, or Ln metals) clusters, or single metal ion (e.g., Mg, Cu, Co, Zn, Mn, Fe, Ni, or Ln metals), and the like.

In an embodiment, the bridging ligands (e.g., coordinating to the metal or metal cluster) and/or the pillar ligands (e.g., linking layers of the MOF, e.g., the SBUs and/or MOPs) can include one or more functional groups (e.g., R1, R2, R3, and/or R4) that can coordinate with the metal(s) and/or link metal containing groups (e.g., some ligands can act as bridging ligands and pillar ligands). It should be noted that the bridging ligands and/or other pillar ligands can include any of the functional groups and compounds described in reference to the porphyrin ligand.

In an embodiment, the pillar ligand can include a porphyrin ligand having the structural formula as shown in FIG. 3.1. In another embodiment, the porphyrin ligand can be used to coordinate with the metal(s) of the SBU. In an embodiment, each of R1, R2, R3, and/or R4 can independently be an organic compound (e.g., moiety) having one or more of the following functional groups: —CO₂H, —CS₂H, —NO₂, —B(OH)₂, —SO₃H, —CN, -tetrazolate, -1,2,3 or 1,2,4-triazolate, -pyrazolate, —PO_{S}H, -pyridyl, and combinations thereof. In an embodiment, the functional groups can be bonded to an organic compound so that they are capable of bonding with the SBU

In an embodiment, each of R1, R2, R3, and/or R4 can independently be an organic compound that can include a saturated or unsaturated aliphatic compound (e.g., alkane, alkene, and the like having 2 to 20 carbons), an aromatic compound (e.g., having 4 to 8 carbons per ring), a heteroaryl compound (e.g., having 4 to 8 atoms per ring), or a compound which includes two or more of aliphatic, aromatic, or heteroaryl characteristics. In an embodiment, each of R1, R2, R3, and/or R4 can independently be an can be an organic compound that can include one or more of the following functional groups: carboxylic acid, amides (including sulfonamide and phosphoramides), sulfinic acids, sulfonic acids, phosphonic acids, phosphates, phosphodiesters, phosphines, boronic acids, boronic esters, borinic acids, borinic esters, nitrates, nitrites, nitriles, nitro, nitroso, thiocyanates, cyanates, azos, azides, imides, imines, amines, acetals, ketals, ethers, esters, aldehydes, ketones, alcohols, thiols, sulfides, disulfides, sulfoxides, sulfones, sulfinic acids, thiones, and thials. In an embodiment, each of R1, R2, R3, and/or R4 can independently be an can be an organic compound that can be: a polycarboxylated ligand (e.g., dicarboxylate ligand, tricarboxylate ligand, or tetra/hexa/octa-carboxylate ligand), a polypyridyl ligand (e.g., dipyridyl ligand, tripyridyl ligand, or tetra/hexa/octa-pyridyl ligand), a polycyano ligand (e.g., dicyano ligand, tricyano ligand, or tetra/hexa/octa-cyano ligand), a polyphosphonate ligand (e.g., diphosphonate ligand, triphosphonate ligand, or tetra/hexa/octa-phosphonate ligand), a polyhydroxyl ligand (e.g., dihydroxyl ligand, trihydroxyl ligand, or tetra/hexa/octa-hydroxyl ligand), a polysulfonate ligand (e.g., disulfonate ligand, trisulfonate ligand, or tetra/hexa/octa-sulfonate ligand), a polyimidazolate, ligand (e.g., diimidazolate ligand, triimidazolate ligand, or tetra/hexa/octa-imidazolate ligand), a polytriazolate (both 1,2,3 and 1,2,4) ligand (e.g., ditriazolate ligand, tritriazolate ligand, or tetra/hexa/octa-triazolate ligands), polytetrazolate ligand (e.g., ditetrazolate ligand, tritetrazolate ligand, or tetra/hexa/octa-tetrazolate ligands), polypyrazolate ligand (e.g., dipyrazolate ligand, tripyrazolate ligand, or tetra/hexa/octa-pyrazolate ligands), and a combination thereof.

In an embodiment, each of R1, R2, R3, and/or R4 can independently be an aromatic dicarboxylic acid moiety, such as an isophthalic acid moiety. In an embodiment, R1 and R3 are H and R2 and R4 are an isophthalic acid moiety. In another embodiment, each of R1, R2, R3, and R4 can be an isophthalic acid moiety. Additional details are provided in the Examples.

In an embodiment, the metalloporphyrin framework structure can be formed by mixing the porphyrin ligand with an SBU or a SBU precursor in a solvent such as DMA, DMF, DEF, DMSO, methanol, ethanol, water, or a combination thereof at a temperature of about 50 to 150° C. It should be noted that the conditions and reagents used can be modified depending upon the metalloporphyrin framework structure formed, the porphyrin ligand, the SBU, and the like. Additional details are provided in the Examples.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

### Example 1

### Brief Introduction:

An unprecedented nanoscopic polyhedral cage-containing metal-metalloporphyrin framework, MMPF-1, has been constructed from a custom designed porphyrin ligand, 5,15-bis(3,5-dicarboxyphenyl)porphine that links Cu₂(carboxylate)₄ moieties. A high density of sixteen open copper sites confined within a nanoscopic polyhedral cage has been achieved, and the packing of the porphyrin cages via an “ABAB” pattern affords MMPF-1 ultramicropores which render it selective towards adsorption of H₂ and O₂ over N₂, and CO₂ over CH₄.

### Discussion:

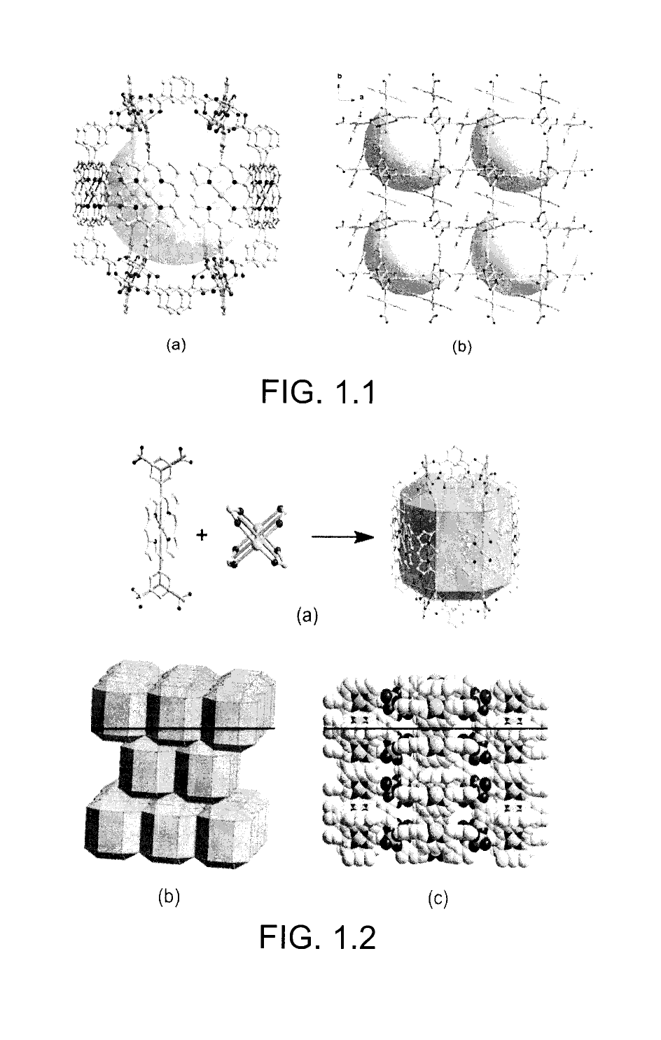

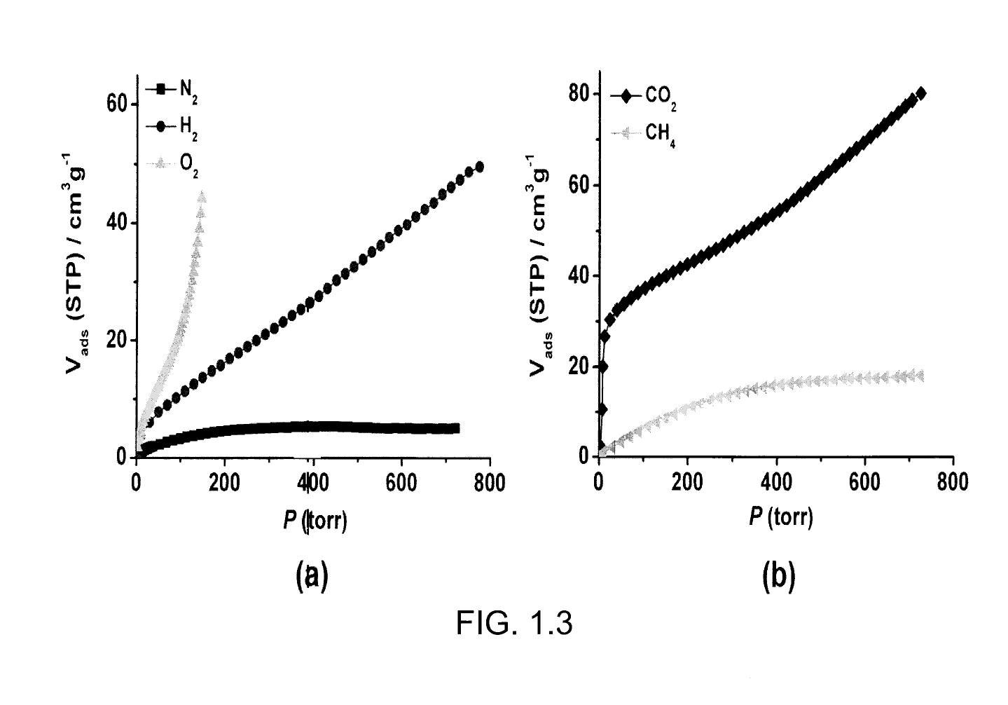

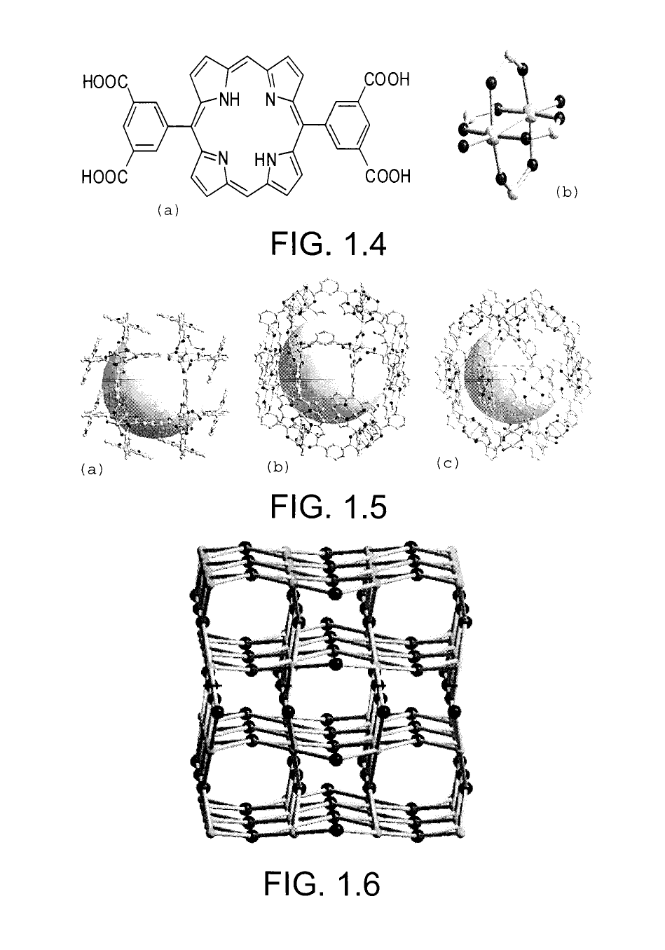

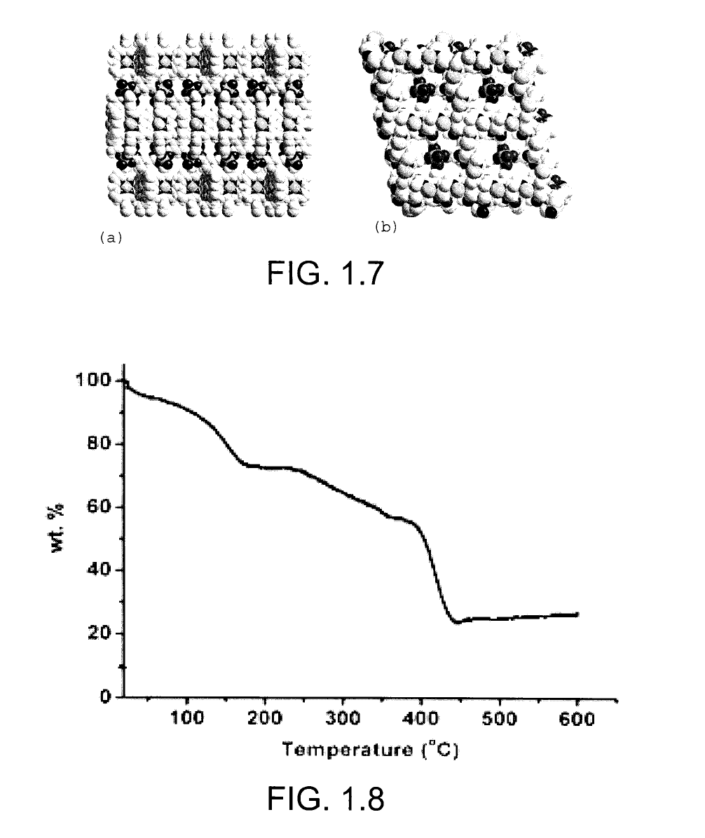

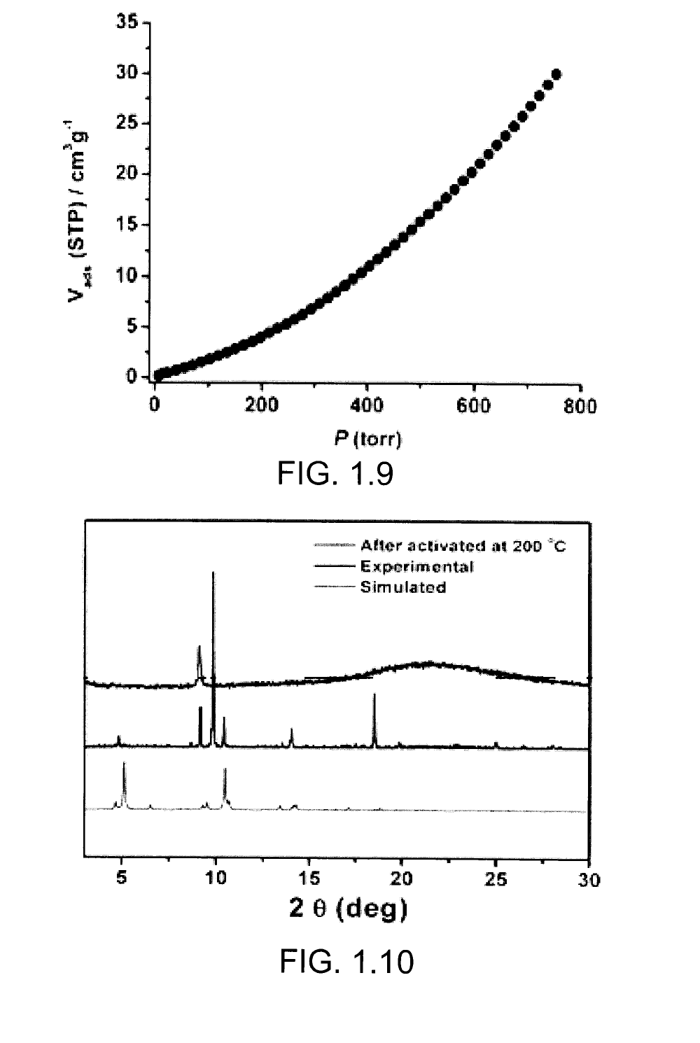

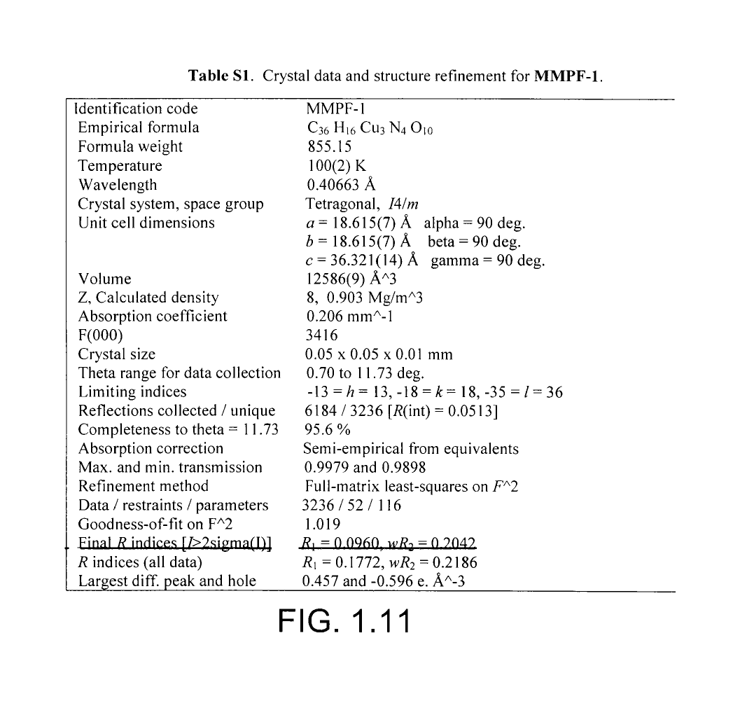

Porphyrins and metalloporphyrins have over decades been intensively studied for a range of applications.¹ The construction of metalloporphyrin-based nanoscopic polyhedral cages affords cage walls rich in π-electron density that can provide favorable interactions with targeted guests.² Such cages also contain multiple active metal centers that could facilitate synergistic interactions with substrates, as exemplified in metalloporphyrin supramolecular materials.²⁻⁴ Concurrently, there has also been an escalating interest in constructing metalloporphyrin-based metal-organic framework (MOF) materials due to their potential applications for gas storage, sensors, and particularly heterogeneous catalysis.⁵ It could be envisioned that if the metalloporphyrin nanoscopic polyhedral cages are built into MOFs, then the 7-electron rich cage walls together with the high density of open metal sites within the confined nanospace would greatly benefit their gas storage and catalytic performances. Although there have been reported a number of metalloporphyrin framework structures in the past decade,^{5,6} polyhedral cage-typed structures have not yet been incorporated into metalloporphyrin-based MOFs. Extensive efforts of utilizing tetrakis(4-carboxyphenyl)porphyrin (tcpp) to assemble with highly symmetric secondary building units (SBUs) of 4- or 6-connectivity to target the polyhedral cage-typed metalloporphyrin framework structure, generally afford 2D layered structures or 3D pillared structures in which the active metal centers within the porphyrin rings are usually blocked^{5c-e, 6h-j}, although some 3D channeled structures with accessible metal centers have been reported recently.^{6d,g,k,l} This is likely to be an artifact of the symmetry of tcpp, which means that it plays the role of a node that is not suitable for the formation of polyhedral cages when connecting highly symmetric SBUs.⁷ Therefore, the incorporation of polyhedral cages into metalloporphyrin-based MOFs remains a challenge and necessitates the custom design of new porphyrin ligands that will be more suited to serve as linkers. In this contribution, we report the first example of such a MOF, which is based upon a metal-organic polyhedron (MOP) formed from a custom designed porphyrin ligand and a judiciously selected SBU. The MOP serves as a supermolecular building block (SBB) that sustains a 3D porous metalloporphyrin framework structure exhibiting a very high density of open metal sites in the confined nanoscopic polyhedral cage.

[M₂(carboxylate)₄] paddlewheel moieties have been widely used for the construction of MOPs as they are ubiquitous in coordination chemistry and their square geometry is versatile in this context.⁸ In particular, vertex-linking of the square SBUs with isophthalate ligands allows the generation of various types of faceted MOPs.⁹ The utilization of these faceted MOPs as SBBs has only recently been employed for the construction of highly porous and symmetrical MOFs by bridging the isophthalates with various organic moieties through their 5-positions, as well exemplified by MOPs based upon isophthalate derivatives and square dicopper paddlewheel SBUs.^{9d, 10} Encouraged by these systems, we anticipate to incorporate the porphyrin moiety into a MOP by designing an isophthalate derived porphyrin ligand, 5,15-bis(3,5-dicarboxyphenyl)porphine (bdcpp), in which a pair of isophthalates are bridged by a porphine macrocycle (FIG. 1.4, Scheme 1a). The assembly of bdcpp with dicopper paddlewheel SBUs (FIG. 1.4, Scheme 1b) afforded an unprecedented 3D porous metalloporphyrin framework, MMPF-1 (MMPF denotes Metal-MetalloPorphyrin Framework) consisting of nanoscopic polyhedral cages with sixteen open copper sites.

MMPF-1 was obtained as dark red block crystals via solvothermal reaction of bdcpp and copper nitrate in dimethylacetamide (DMA) at 85° C. Single-crystal X-ray crystallographic studies¹¹ conducted using synchrotron radiation at the Advanced Photon Source, Argonne National Laboratory revealed that MMPF-1 crystallizes in the space group l4/m, and consists of dicopper paddlewheel SBUs linked by bdcpp ligands.

In the bdcpp ligand, the four carboxylate groups and the two phenyl rings of the isophthalate moieties are almost coplanar, whereas the dihedral angle between the porphyrin ring and the phenyl rings is 69.2°. Sixteen bdcpp ligands connect eight paddlewheel SBUs to form a nanoscopic cage. Four dicopper paddlewheel SBUs are bridged by four isophthalate moieties of four different bdcpp ligands to form the top of the cage; they are pillared to four dicopper paddlewheel SBUs at the bottom of the cage through eight different bdcpp ligands (FIG. 1.1a). The porphyrin macrocycle of the bdcpp ligand is metallated in-situ by Cu(II) ion that is free of coordinated solvent molecules probably due its unavailability for axial ligation,^{6a} thus leaving both the distal and proximal positions open. The porphyrin ring of each bdcpp is in close contact with two adjacent porphyrin rings, one of which lies parallel (2.850 Å between an H atom of one porphyrin ring and the plane of the porphyrin ring of an adjacent bdcpp ligand) whereas the other lies orthogonal (2.554 Å between an H atom of one porphyrin ring and the plane of the adjacent porphyrin). The cage contains three types of window: there are two square windows formed by four dicopper paddlewheel SBUs through four isophthalate moieties with dimensions of 8.070 Å×8.070 Å (atom to atom distance) (FIG. 1.5a); there are eight rectangular windows formed by two dicopper paddlewheel SBUs and two half porphyrin rings via three isophthalate motifs with dimensions of 7.065 Å×7.181 Å (FIG. 1.5b); there are eight triangular windows formed by linking one dicopper paddlewheel SBU with two half porphyrin rings through two isophthalate motifs with dimensions of 6.979 Å×6.979 Å×7.640 Å (FIG. 1.5c). In each cage, there are eight open copper sites associated with the porphyrin rings of the bdcpp ligands and eight open copper sites from dicopper paddlewheel SBUs that are activated by thermal liberation of aqua ligands. The distance between copper atoms within the opposite porphyrin rings is 18.615 Å whereas copper atoms in adjacent porphyrin rings are separated by 7.571 Å and 7.908 Å; open copper sites from two opposite dicopper paddlewheel SBUs at the top and the bottom of the cage lie 16.170 Å apart whereas open copper sites from adjacent SBUs lie 8.070 Å apart. The volume of the cage is ˜2340 Å³, and it is filled with highly disordered solvent molecules of DMA and water that cannot be mapped by single-crystal X-ray studies even using a synchrotron radiation source. All sixteen open copper sites point toward the center of the cage, an unprecedentedly high density of open metal sites in a nanoscopic cage (˜7 open metal sites/nm³) (FIG. 1.1a).

If one connects the centers of all isophthalate phenyl rings and the centers of the eight paddlewheels, the cage can be depicted as a polyhedron, which has 24 vertices, 26 faces, and 48 edges (FIG. 1.2a). In view of its similar shape to the rhombicuboctahedron connected by 24 isophthalates and 12 paddlewheels in MOPs and some MOFs,^{9d,10b} this polyhedron can be also described as an irregular rhombicuboctahedron. These irregular rhombicuboctahedra serve as SBBs to extend in the ab plane (FIG. 1.1b) and then pack along c via “ABAB” stacking to form an overall 3D structure (FIG. 1.2b). Topologically, MMPF-1 can be described as a 3D 4-connected net possessing lvt-like topology (FIG. 1.6).¹²

Due to the ABAB packing, the two square windows and eight triangular windows of each nanoscopic cage are totally blocked, whereas the eight rectangular windows are eclipsed with remaining apertures of ˜3.4 Å×3.5 Å (Van der Waals distance), as can be viewed from the [1 0 0] (FIG. 1.2c), [0 1 0] (FIG. 1.7a), and [1 1 1] directions (FIG. 1.7b). These tiny apertures could let very small molecules like water (kinetic diameter: 2.64 Å) pass through but could hardly allow the DMA solvent molecules (molecule size: ˜5.24 Å×4.52 Å×4.35 Å) that are trapped in the irregular rhombicuboctahedral cages to escape.

Thermogravimetric analysis (TGA) of the fresh MMPF-1 sample (FIG. 1.8) reveals that the first weight loss of 25.95% (calculated: 25.33%) from 20° C. to ˜170° C. corresponds to loss of two DMA molecules adsorbed on the surface, six H₂O guest molecules trapped in the irregular rhombicuboctahedral cages, and the two terminal aqua ligands liberated from the copper paddlewheel SBUs. A steady plateau from ˜170° C. to ˜240° C. is followed by the loss of two DMA guest molecules trapped in the cages (found: 14.34%; calculated: 13.32%), presumably accompanied by decomposition of the copper paddlewheel SBUs¹³ at ˜360° C. The loss of bdcpp ligands starts from ˜370° C. and finishes at ˜450° C., and results in complete collapse of the MMPF-1 framework.

The tiny pore sizes of MMPF-1 which are a result of the “ABAB” packing of the irregular rhombicuboctahedral cages prompted us to evaluate its performance as a selective gas adsorbent. A freshly prepared MMPF-1 sample was washed with methanol and thermally activated at 120° C. under dynamic vacuum before gas adsorption measurements. N₂ adsorption isotherms were collected at 77 K, and as shown in FIG. 1.3a, a very limited amount of N₂ (5 cm³/g) is adsorbed on the external surface of MMPF-1 at 760 torr. In contrast, a larger amount of H₂ uptake (50 cm³/g) is observed under the same condition, and a substantial uptake of 45 cm³/g is also found for O₂ at its saturation pressure of 154 torr at 77K. Gas adsorption studies at 195 K indicated that MMPF-1 can uptake a large amount of CO₂ (80 cm³/g) at 760 torr, which is much higher than CH₄ (18 cm³/g). The interesting molecular sieving effect observed for MMPF-1 can be attributed to its small aperture sizes of ˜3.5 Å, which exclude larger gas molecules of N₂ and CH₄ with kinetic diameters of 3.64 Å and 3.8 Å respectively but allow the entry of smaller gas molecules of H₂ (kinetic diameter: 2.89 Å), O₂ (kinetic diameter: 3.46 Å), and CO₂ (kinetic diameter: 3.3 Å). The selective adsorption of H₂ and O₂ over N₂, and CO₂ over CH₄ observed for MMPF-1 is rare;¹⁴ to the best of our knowledge, it represents the first example reported in metalloporphyrin-based MOFs. Our attempts to remove the DMA guest molecules trapped in the nanoscopic cages by activating MMPF-1 at 200° C. in order to improve its uptake capacities of H₂ and CO₂, unfortunately led to partial collapse of the framework as evidenced by significant decrease of CO₂ uptake (FIG. 1.9) and the loss of crystallinity (FIG. 1.10), which indicates its modest thermal stability.

In summary, by employing the SBB strategy, an unprecedented three-dimensional porous metal-metalloporphyrin framework (MMPF) that consists of nanoscopic rhombicuboctahedral cages with a high density of sixteen open copper sites has been prepared based upon the custom designed bdcpp ligands that link copper paddlewheel SBUs. The “ABAB” packing of the rhombicuboctahedral cages in MMPF-1 constricts its pore size, which facilitates selective adsorption of H₂ and O₂ over N₂, and CO₂ over CH₄. Considering the high density of open metal sites confined within a nanoscopic cage, ongoing work in our laboratories will focus upon designing new porphyrin ligands to construct cage-containing porous metalloporphyrin frameworks with larger pore sizes, and to explore them for applications in gas storage, sensors, and particularly heterogeneous catalysis for small molecules.

REFERENCES, each of which is incorporated herein by reference.
(1) Kadish, K. M.; Smith, K. M.; Guilard, R.; Eds. The Porphyrin Handbook; Academic Press: San Diego, 2000-2003.
(2) (a) Nakamura, Y.; Aratani, Naoki; Osuka, A. Chem. Soc. Rev. 2007, 36, 831-845; (b) Beletskaya, I.; Tyurin, V. S.; Tsivadze, A. Y.; Guilard, R.; Stern C. Chem. Rev. 2009, 109, 1659-1713.
(3) Drain, C. M.; Varotto, A.; Radivojevic, I. Chem. Rev. 2009, 109, 1630-1658.
(4) (a) Song, J.; Aratani, N.; Shinokubo, H.; Osuka, A. J. Am. Chem. Soc. 2010, 132, 16356-16357; (b) Meng, W.; Breiner, B.; Rissanen, K.; Thoburn, J. D.; Clegg, J. K.; Nitschke, J. R. Angew. Chem. Int. Ed. 2011, 50, 3479-3483; (c) O'Sullivanl, M. C.; Sprafke1, J. K.; Kondratuk, D. V.; Rinfray, C.; Claridge, T. D. W.; Saywell, A.; Blunt, M. O.; O'Shea, J. N.; Beton, P. H.; Malfois, M. Anderson, H. L. Nature, 2011, 469, 72-75.
(5) (a) Kosal, M. E.; Suslick, K. S. J. Solid State Chem. 2000, 152, 87-98; (b) Suslick, K. S.; Bhyrappa, P.; Chou, J.-H.; Kosal, M. E.; Nakagaki, S.; Smithenry, D. W.; Wilson, S. R. Acc. Chem. Res. 2005, 38, 283-291; (c) Goldberg, I. Chem. Commun. 2005, 1243-1254; (d) Goldberg, I. Cryst Eng Comm. 2008, 10, 637-645; (e) DeVries, L. D.; Choe, W. J. Chem. Crystallogr. 2009, 39, 229-240.
(6) (a) Abrahams, B. F.; Hoskins, B. F.; Michail, D. M.; Robson, R. Nature, 1994, 369, 727-729; (b) Kumar, R. K.; Goldberg, I. Angew. Chem. Int. Ed. 1998, 37, 3027-3040; (c) Lin, K.-J. Angew. Chem. Int. Ed. 1999, 38, 2730-2732; (d) Kosal, M. E.; Chou, J.-H.; Wilson, S. R.; Suslick, K. S. Nat. Mater. 2002, 1, 118-121; (e) Smithenry, D. W.; Wilson, S. R.; Suslick, K. S. Inorg. Chem. 2003, 42, 7719-7721; (f) Ohmura, T.; Usuki, A.; Fukumori, K.; Ohta, T.; Ito, M.; Tatsumi, K. Inorg. Chem. 2006, 45, 7988-7990; (g) Shultz, A. M.; Farha, O. K.; Hupp, J. T.; Nguyen, S. T. J. Am. Chem. Soc. 2009, 131, 4204-4205; (h) Choi, E.-Y.; Wray, C. A.; Hu, C.; Choe, W. Cryst Eng Comm. 2009, 11, 553-555; (i) Choi, E.-Y.; Barron, P. M.; Novotny, R. W.; Son, H.-T.; Hu, C.; Choe, W. Inorg. Chem. 2009, 48, 426-428; (j) Chung, H.; Barron, P. M.; Novotny, R. W.; Son, H.-T.; Hu, C.; Choe, W. Crystal Growth & Design 2009, 9, 3327-3332; (k) Barron, P. M.; Wray, C. A.; Hu, C.; Guo, Z.; Choe, W. Inorg. Chem. 2010, 49, 10217-10219; (I) Farha, O. K.; Shultz, A. M.; Sarjeant, A. A.; Nguyen, S. T.; Hupp, J. T. J. Am. Chem. Soc. 2011, 133, 5652-5655.
(7) O'Keeffe, M. Chem. Soc. Rev. 2009, 38, 1215-1217.
(8) (a) Z. Ni, M. O'Keeffe, O. M. Yaghi, Angew. Chem. Int. Ed., 2008, 47, 5136-5147; (b) Li, J.-R.; Yakovenko, A.; Lu, W.; Timmons, D. J.; Zhuang, W.; Yuan, D.; Zhou, H.-C., J. Am. Chem. Soc. 2010, 132, 17599-17610; (c) (d) Li, J.-R.; Zhou, H.-C., Nature Chem. 2010, 2, 893-898; (e) Li, J.-R., Zhou, H.-C., Angew. Chem. Int. Ed., 2009, 48, 8465-8468.
(9) (a) Ke, Y.; Collins, D. J.; Zhou, H.-C. Inorg. Chem. 2005, 44, 4154-4156; (b)H. Furukawa, J. Kim, K. E. Plass, O. M. Yaghi, J. Am. Chem. Soc., 2006, 128, 8398-8399; (c) Perry, J. J.; Kravtsov, V. C.; McManus, G. J.; Zaworotko, M. J. J. Am. Chem. Soc. 2007, 129, 1076-1077; (d) Perry, J. J.; Perman, J. A.; Zaworotko, M. J. Chem. Soc. Rev. 2009, 38, 1400-1417.
(10) (a) Nouar, F.; Eubank, J. F.; Bousquet, T.; Wojtas, L.; Zaworotko, M. J.; Eddaoudi, M. J. Am. Chem. Soc. 2008, 130, 1833-1835; (b) Cairns, A. J.; Perman, J. A., Wojtas, L.; Kravtsov, V. C.; Alkordi, M. H.; Eddaoudi, M.; Zaworotko, M. J. J. Am. Chem. Soc. 2008, 130, 1560-1561.
(11) X-ray crystal data for MMPF-1: C₃₆H₁₆Cu₃N₄O₁₀, fw=855.15, tetragonal, l4/m, a=18.615 (7) Å, b=18.615 (7) Å, c=36.321 (1) Å, V=12586 (9) Å³, Z=8, T=100 K, ρ_{calcd}=0.903 g/cm³, R₁ (I>2σ(l))=0.0960, wR₂ (all data)=0.2042.
(12) Perman, J. A.; Cairns, A. J.; Wojtas, L.; Eddaoudi, M.; Zaworotko, M. J. Cryst Eng Comm 2011, 13, 3130-3133
(13) (a) Chui, S. S.-Y.; Lo, S. M.-F.; Charmant, J. P. H.; Orpen, A. G.; Williams, I. D. A. Science, 1999, 283, 1148-1150; (b) Schlichte, K.; Kratzke, T.; Kaskel, S. Micropor. Mesopor. Mater. 2004, 73, 81-88; (c) Yuan, D.; Zhao, D.; Timmons, D. J.; Zhou, H.-C. Chem. Sci. 2011, 2, 103-106.
(14) (a) Dybtsev, D. N.; Chun, H.; Yoon, S. H.; Kim, D.; Kim, K. J. Am. Chem. Soc. 2004, 126, 32-33; (b) Chen, B.; Ma, S.; Zapata, F.; Fronczek, F. R.; Lobkovsky, E. B.; Zhou, H.-C., Inorg. Chem., 2007, 46, 1233-1236; (c) Ma, S.; Wang, X.-S.; Collier, C. D.; Manis, E. S.; Zhou, H-C., Inorg. Chem. 2007, 46, 8499-8501; (d) Ma, S.; Wang, X.-S.; Yuan, D. Zhou, H. C. Angew Chem. Int. Ed., 2008, 47, 4130-4133; (e) Ma, S. Pure & Appl. Chem., 2009, 81, 2235-2251; (f) Li, J.-R.; Kuppler, R. J.; Zhou, H.-C. Chem. Soc. Rev. 2009, 38, 1477-1504.

### SUPPLEMENTAL MATERIAL FOR EXAMPLE 1

### Synthesis of 5,15-bis(3,5-dicarboxyphenyl)porphine (bdcpp)

bdcpp ligand was prepared according to the method described in literature.¹ A mixture of dipyrrolemethane (292 mg, 2 mmol) and dimethyl 5-formylisophthalate² (444 mg, 4 mmol) and molecular sieves (4 A, 0.600 g) in CHCl₃ (300 ml) was bubbled with N₂ for 20 min, then BF₃.Et₂O (0.2 mL) was added. The reaction vessel was shaded from the ambient light and left to stir at room temperature for 3 h, and 2,3-dichloro-5,6-dicyano-1,4-benzoquinone (DDQ) (547 mg, 2.4 mmol) was added as powder at one time. The resulting solution was stirred further for 30 minutes. The reaction mixture was loaded directly on the top of a silica gel column and eluted with CH₂Cl₂ to obtain the ester, which was hydrolyzed to afford the pure compound. Yield: ˜12.5 mg, ˜1%. ¹HNMR (250 MHz, DMSO): δ 10.69 (s, 2H), 9.69 (d, J=4.5 Hz, 4H), 9.03 (d, J=4.75 Hz, 4H), 8.95 (s, 6H), −3.29 (s, 2H).

### Synthesis of MMPF-1:

A mixture of bdcpp (0.002 g), Cu(NO₃)₂.2.5H₂O (0.005 g), and 1.5 mL dimethylacetamide (DMA) was sealed in a Pyrex tube under vacuum and heated to 85° C. for 24 hours. The resulting dark red block crystals were washed with DMA to give pure MMPF-1 with a formula of Cu₃(bdcpp)(H₂O)₂.4DMA.6H₂O (yield: 65% based on bdcpp; Elemental analysis: Calculated (%): C, 47.61; H, 4.92; N, 8.54. Found (%): C, 49.86; H, 4.94; N, 8.72).

### Single-Crystal X-Ray Diffraction Studies of MMPF-1:

The X-ray diffraction data were collected using synchrotron radiation, λ=0.40663 Å, at Advanced Photon Source, Chicago Ill. Indexing was performed using APEX2³ (Difference Vectors method). Data integration and reduction were performed using SaintPlus 6.01⁴. Absorption correction was performed by multi-scan method implemented in SADABS.⁵ Space groups were determined using XPREP implemented in APEX2.³ The structure was solved using SHELXS-97 (direct methods) and refined using SHELXL-97 (full-matrix least-squares on F²) contained in APEX2³ and WinGX v1.70.01⁶⁻⁹ programs packages. Despite of using synchrotron source and trying several crystals from different batches, diffraction experiment resulted in low quality diffraction data (lack of high angle reflections). This can be attributed to the presence of the ligand/solvent disorder and to the presence of bad quality, multiply twinned crystals. Due to the low resolution of the data, C,N,O atoms were refined with isotropic displacement parameters and disordered ligand moiety was refined using distance restraints. Hydrogen atoms were placed in geometrically calculated positions and included in the refinement process using riding model with isotropic thermal parameters: Uiso(H)=1.2Ueq(-CH). The contribution of disordered solvent molecules was treated as diffuse using Squeeze procedure implemented in Platon program.^{10,11} Crystal data and refinement conditions are shown in FIG. 1.11, Table S1. (see FIGS. 1.5 to 1.10 for additional details)

### Gas Adsorption Experiments.

Gas adsorption isotherms of MMPF-1 were collected using the surface area analyzer ASAP-2020. Before the measurements, the freshly prepared samples were washed with methanol, and then activated under dynamic vacuum at 120° C. for two hours. N₂, O₂, and H₂ gas adsorption isotherms were measured at 77 K using a liquid N₂ bath, and CO₂ and CH₄ gas adsorption isotherms were measured at 195 K using an acetone-dry ice bath.

References, each of which is incorporated herein by reference:
1. Lindsey, J. S.; Wagner, R. W. J. Org. Chem. 1989, 54, 828.
2. Rochford, J.; Galoppini, E. Langmuir 2008, 24, 5366.
3. Bruker, 2010, APEX2). Bruker AXS Inc., Madison, Wis., USA.
4. Bruker, 2009, SAINT. Data Reduction Software. Bruker AXS Inc., Madison, Wis., USA.
5. Sheldrick, G. M. 2008, SADABS. Program for Empirical Absorption. Correction. University of Gottingen, Germany.
6. Farrugia L. J. Appl. Cryst. 1999, 32, 837.
7. Sheldrick, G. M. 1997, SHELXL-97. Program for the Refinement of Crystal.
8. Sheldrick, G. M. Acta Cryst. 1990, A46, 467.
9. Sheldrick, G. M. Acta Cryst. 2008, A64, 112.
10. Spek, T. L. Acta Cryst. 1990, A46, 194-201.
11. Spek, T. L. Acta Cryst. 1990, A46, c34.

### EXAMPLE 2

### Brief Description:

A porous metal-metalloporphyrin framework, MMPF-2, has been constructed from a custom-designed octatopic porphyrin ligand, tetrakis(3,5-dicarboxyphenyl)porphine, that links a distorted cobalt trigonal prism SBU; MMPF-2 possesses permanent microporosity with the highest surface area of 2037 m²/g among reported porphyrin-based MOFs, and demonstrates a high uptake capacity of 170 cm³/g CO₂ at 273 K and 1 bar.

### Discussion:

As an important type of biologically-relevant macrocycles, porphyrins and metalloporphyrins have been of intense research interests in the past decades.¹ One of their important features lies in the characteristic diversity which can be obtained through the addition of a variety of central metal entities, or via the introduction of functional peripheral substituents.² This has afforded them as a class of versatile materials for a range of applications,³ as particularly witnessed by the rapid progress in the development of porphyrin/metalloporphyrin supramolecular materials.⁴

Concurrently, there has also been an escalating interest in constructing porphyrin/metalloporphyrin-based metal-organic framework (MOF) materials due to their potential applications for gas storage, artificial light harvesting system, heterogeneous catalysis, etc.⁵ The first porphyrin-based MOF dates back to as early as 1991 as reported by Robson et al.,⁶ and since then 94 two- or three-dimensional porphyrin-based MOF structures have been reported (see ESI for complete references). Although the development of porphyrin-based MOFs as functional materials particularly as zeolite analogues for size and/or shape-selective heterogeneous catalysis as well gas storage/separation,^{5,7} has been pursued over two decades, limited progress has thus far been made in this research area. It has been recognized that porphyrin-based MOFs are notoriously apt to collapse upon the removal of guest solvent molecules,⁸ and the low surface areas together with framework fragility have afforded them poor capability for gas storage⁹ as well as moderate heterogeneous catalysis performance with either exterior surface catalysis^{5c,10} or lack of recyclability.⁸ Indeed, only 13 of those 94 porphyrin-based MOF structures have been reported to possess porosity as evidenced by gas sorption studies (Table S1);^{5b,8,9,11}and the highest surface area (Langmuir or NLDFT surface area) value reported thus far is merely ˜1000 m²/g,^{8b,11b} which is much less than its predicted value, indicating the possible collapse of majority of the framework. Hence, the construction of robust high-surface-area porphyrin-based MOFs remains a grand challenge to develop them as functional materials for various applications particularly for gas storage and catalysis.

To address this challenge, herein we report the approach of combined use of a custom-designed multitopic porphyrin ligand and a robust secondary building unit (SBU), which is expected to stabilize the MOF structure and preserve its permanent porosity upon removal of guest solvent molecules thus affording superior gas storage performances compared to existing porphyrin-based MOFs. To achieve this goal, we designed a novel octatopic porphyrin ligand, tetrakis(3,5-dicarboxyphenyl)porphine (H₁₀tdcpp) (Scheme 1a), and linked it to a distorted cobalt trigonal prism SBU (Scheme 1b) generated in situ to afford a robust (6, 8, 8)-connected MOF with a new topology of msq,¹² which we denote MMPF-2 (MMPF represents metal-metalloporphyrin framework). As expected, MMPF-2 possesses the highest surface area of 2037 m²/g among reported porphyrin-based MOFs, and the high surface area in combination with the high density of open cobalt centers of the porphyrin macrocyles that are rigidly located in a “face-to-face” configuration to form the channel walls also affords it interesting CO₂ capture performances.

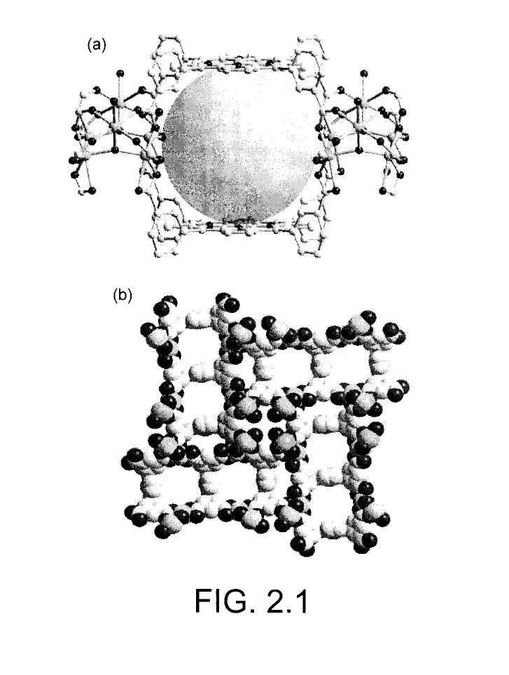

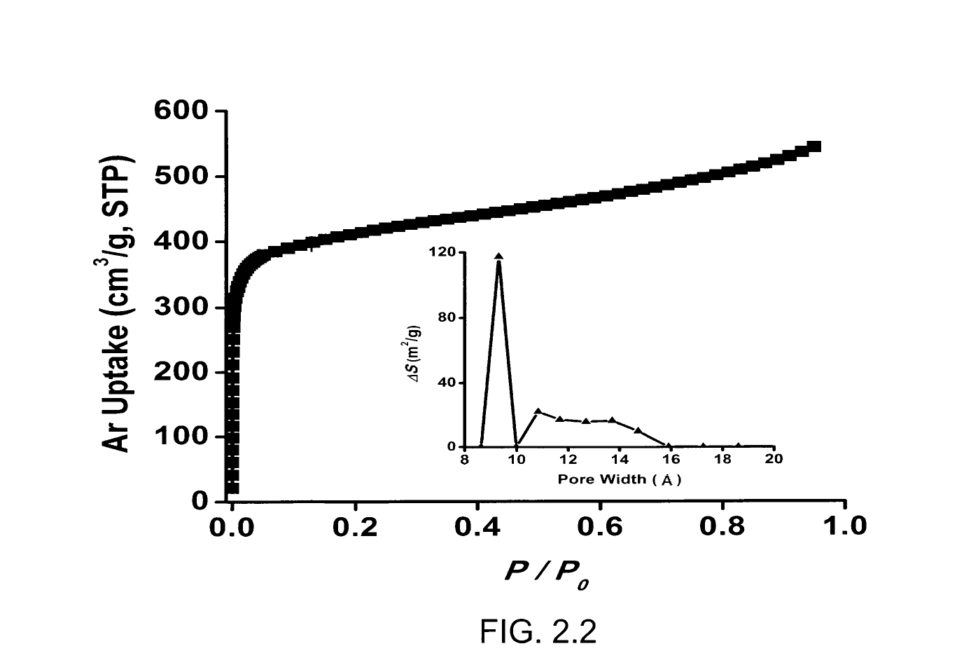

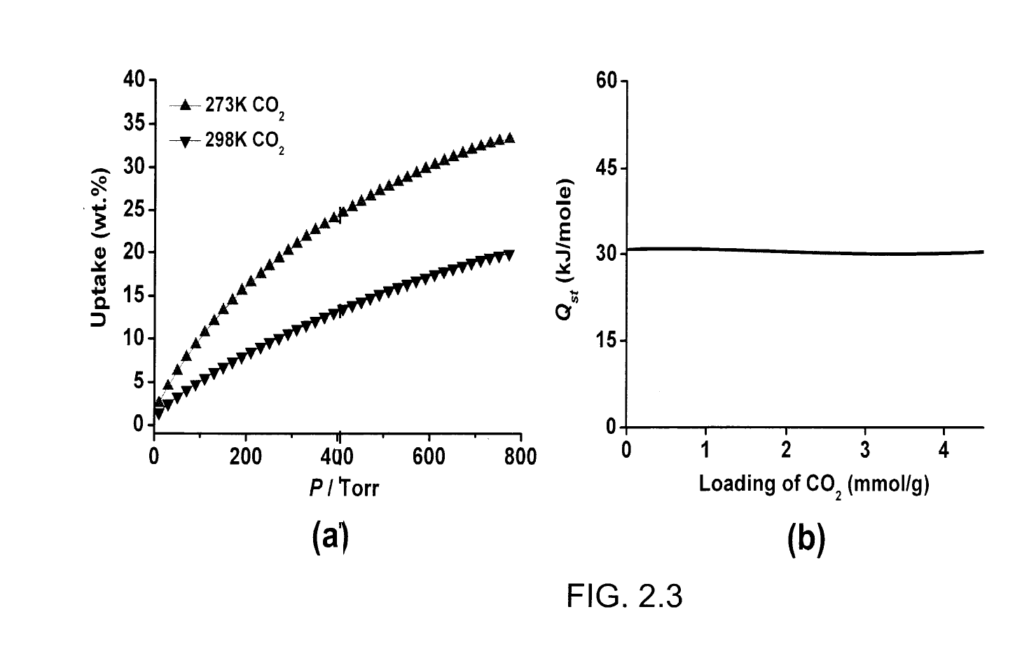

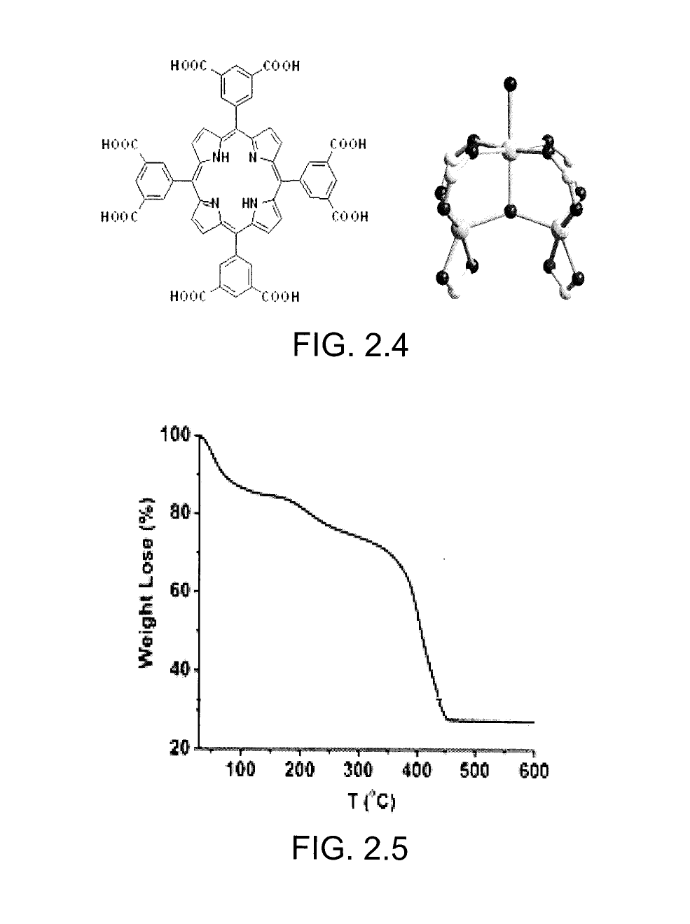

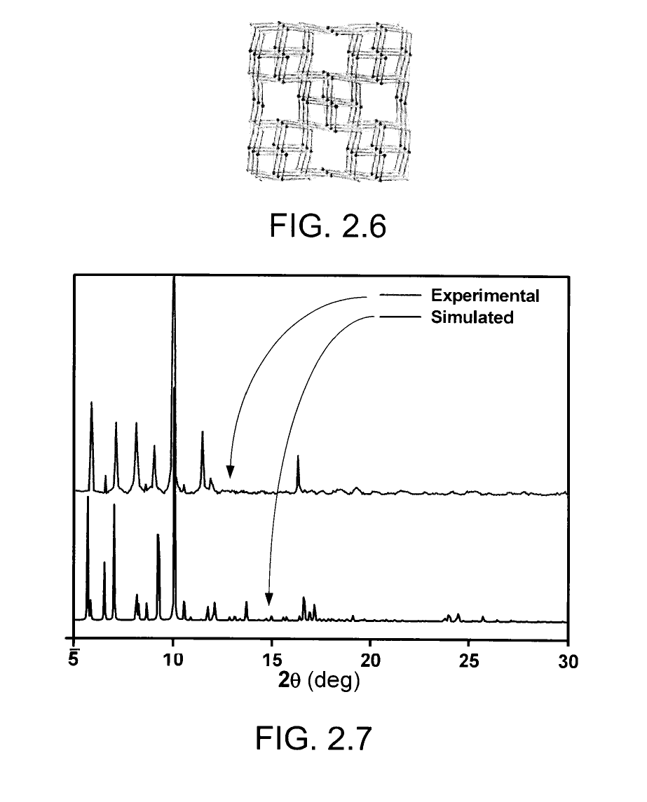

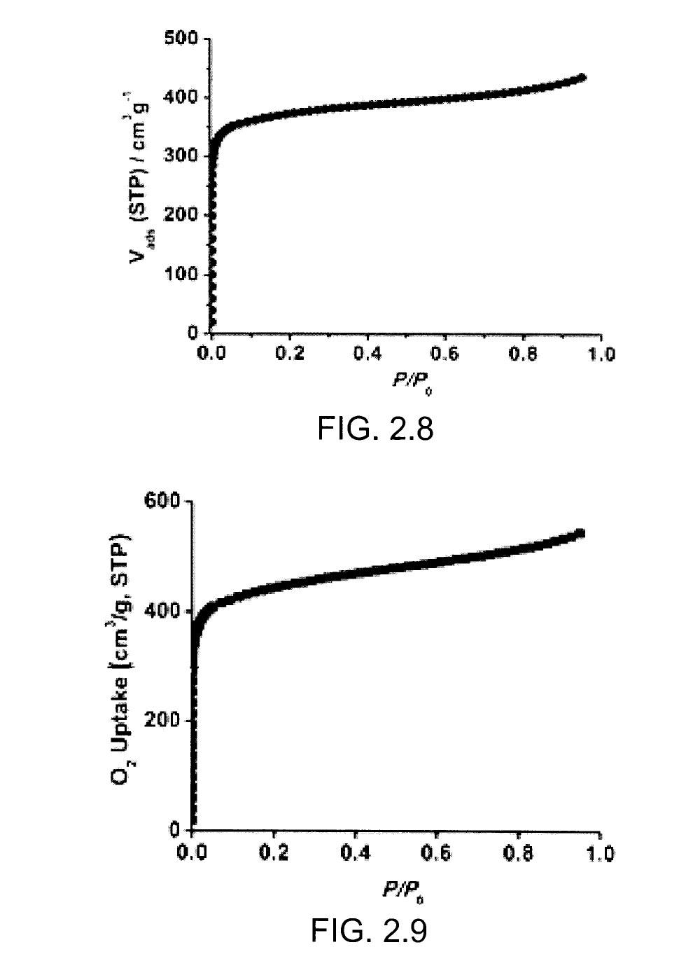

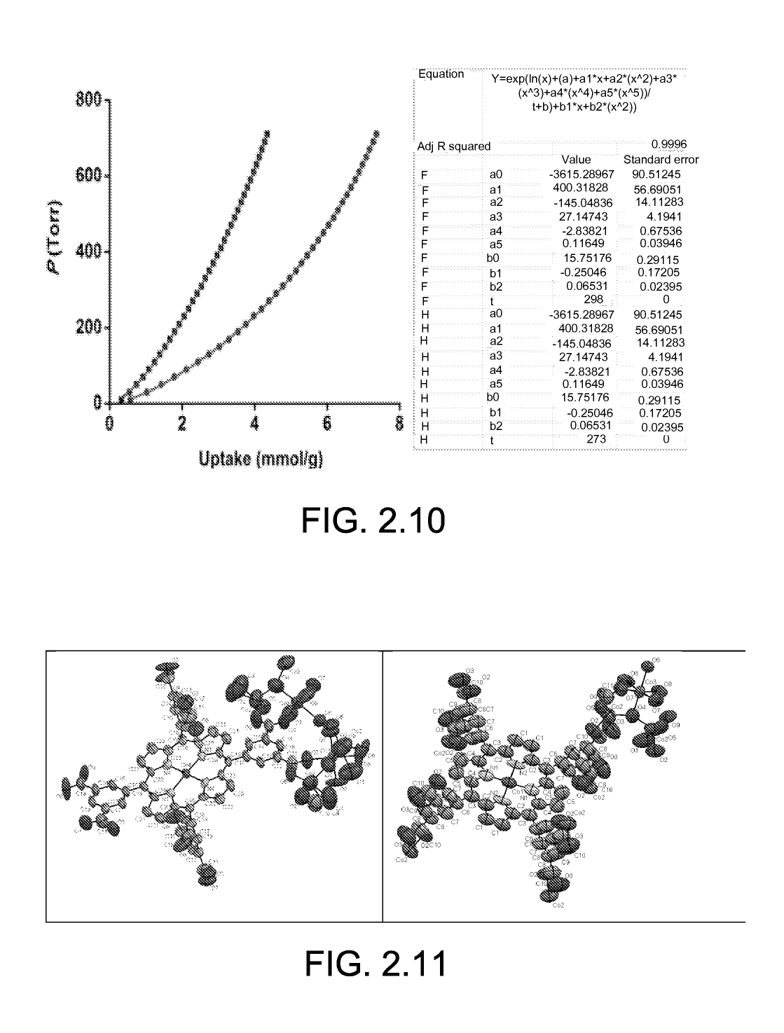

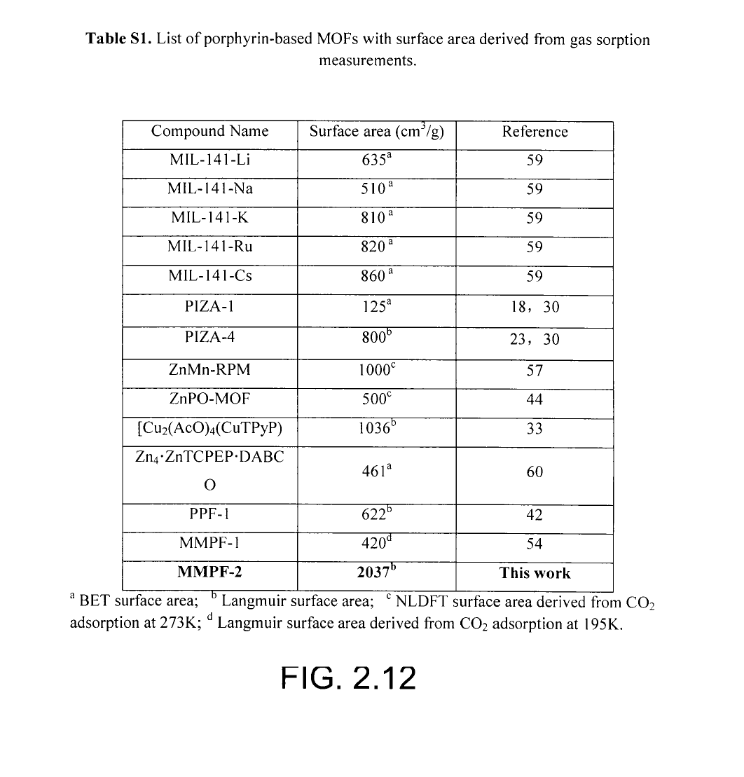

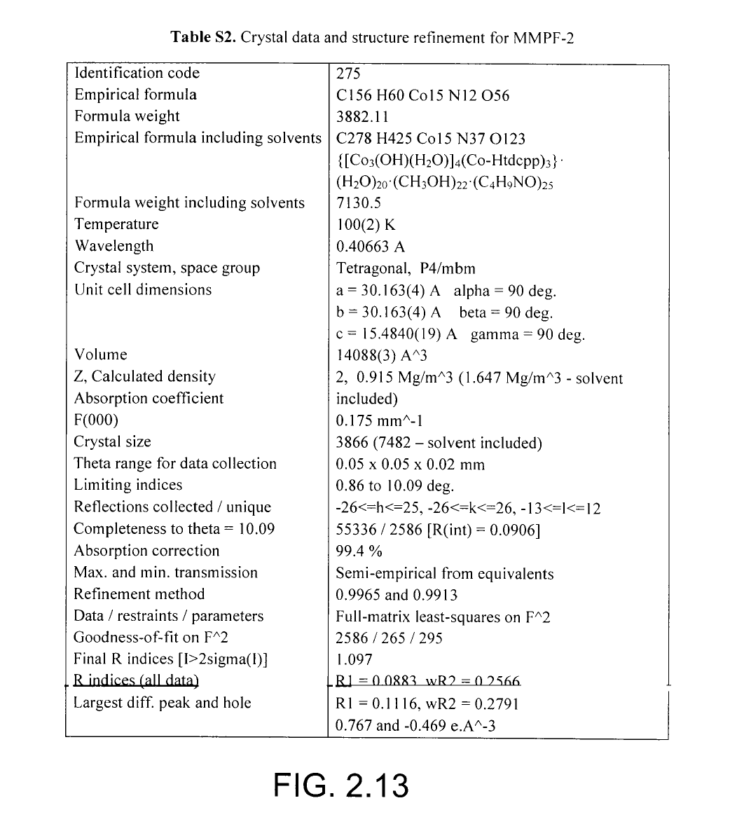

Crystals of MMPF-2 were formed via solvothermal reaction of the H₁₀tdcpp and Co(NO₃)₂.6H₂O in dimethylacetamide (DMA) at 115° C. The product was isolated as dark red block crystals of {[Co(II)₃(OH)(H₂O)]₄(Co(II)tdcp)₃}.(H₂O)₂₀.(CH₃OH)₂₂.(DMA)₂₅ at 60% yield. The overall formula was determined by X-ray crystallography, elemental analysis, and thermogravimetric analysis (TGA) (FIG. 2.5).

Single-crystal X-ray studies conducted using synchrotron microcrystal diffraction at the Advanced Photon Source, Argonne National Laboratory, revealed that MMPF-2 crystallizes in the tetragonal space group P₄lmbm. It adopts a rare distorted cobalt trigonal prism SBU,¹³ in which three cobalt atoms bridged by the μ₃-OH group connect with six carboxylate groups from six different tdcpp ligands (Scheme 1b). The distorted cobalt trigonal prism SBU¹⁴ of MMPF-2 exhibits four carboxylate groups that are bi-dentate and two that are mono-dendate; only one cobalt atom is six coordinate while the other two cobalt atoms are five coordinate. Each SBU links six tdcpp ligands which are divided into two types according to the mono/bi-chelation modes of the carboxylate groups, and every tdcpp ligand connects with eight SBUs. If one assumes the SBU to be a six connected node and the tdcpp ligand to be an eight connected vertex, topologically MMPF-2 possesses an unprecedented (6, 8, 8)-connected trinodal net with a new topology of msq (vertex symbol: (4¹³·6²)⁴(4²⁰·6⁸)²(4²⁴·6⁴)⁴) (FIG. 2.6).¹⁵

In the tdcpp ligand, four isophthalate moieties are almost perpendicular to the porphyrin plane so that four carboxylate groups point upward and the other four point downward. This allows the tdcpp ligand featuring mono-chelated carboxylate groups to rigidly bridge two other tdcpp ligands via eight distorted cobalt trigonal prism SBUs, resulting in porphyrin macrocycles located in a “face-to-face” configuration with the distance between two cobalt centers within a porphyrin rings of 10.262 Å (atom to atom distance) (FIG. 2.1a). Every fourth SBU is bridged by four isophthalate moieties and propagates along the c direction to form a small hydrophilic square channel with all the terminal aqua ligands from SBUs pointing toward the channel center (FIG. 2.1b); the distance between two opposite water molecules in the channel is 5.388 Å and that between two neighboring ones is 3.810 Å. The square hydrophilic channel is surrounded by four sets of three cofacial metalloporphyrin rings, which extend along c direction to form two rectangular channels with a size of 10.046 Å×10.099 Å. A third channel surrounding it is enclosed by two SBUs, one tdcpp ligand, and one isophthalate moiety and exhibits dimensions of 6.204 Å×7.798 Å (FIG. 2.1b). Both the distal and proximal positions of the cobalt atoms within the porphyrin macrocycles are open toward the channels, allowing substrate or guest molecules to bind. The solvent accessible volume of MMPF-2 calculated using PLATON is 60.1%.¹⁶

TGA studies of the fresh MMPF-2 sample (FIG. 2.5) reveals almost a continuous weight loss of ˜30% from 30 to ˜360° C. corresponding to loss of guest solvent molecules and terminal aqua ligands liberated from distorted cobalt trigonal prism SBUs, which is closely followed by the loss of tdcpp ligands till ˜450° C. leading to complete collapse of the MMPF-2 framework.

One of the most challenging issues for porphyrin-based MOFs lies in the preservation o porosity upon removal of guest solvent molecules.⁸ To assess the permanent porosity of MMPF-2, we performed gas sorption measurements on the activated MMPF-2 sample. As shown in FIG. 2.2, the Ar adsorption isotherm at 87 K reveals that MMPF-2 exhibits an uptake capacity of 545 cm³/g at the saturation pressure with typical type-I sorption behavior, as expected for microporous materials. Derived from the Ar adsorption data, MMPF-2 has a Langmuir surface area (P/P₀=0.9) of 2037 m²/g (BET surface area (P/P₀=0.02-0.2), 1410 m²/g), which is the highest among reported porphyrin-based MOFs (Table S1).^{5b,8,9,11} The measured pore volume of MMPF-2 is 0.61 cm³/g, which is consistent with the solvent accessible volume of 60.1% and also matches the calculated value of 0.63 cm³/g,¹⁶ highlighting the robustness of its framework. The high surface area of MMPF-2 was further confirmed by N₂ adsorption at 77 K (FIGS. 2.8) and O₂ adsorption at 87 K (FIG. 2.9), both of which reveal similar surface area values. Density function theory (DFT) pore size distribution analysis based on the Ar adsorption data at 87 K revealed that the pore size of MMPF-2 is predominantly around 9.5 Å (FIG. 2.2 insert), which is close to the cofacial metalloporphyrin channel size of ˜10 Å observed crystallographically.

We investigated CO₂ uptake performances of MMPF-2. The CO₂ adsorption isotherm measured at 273 K indicates that MMPF-2 has an uptake capacity of 33.4 wt. % (or 170 cm³/g, or 7.59 mmol/g) (FIG. 2.3a) at 760 torr, which is comparable to the highest value of 38.5 wt. % for the porous MOF, SNU-5 under the same condition despite its much lower surface area (2037 m²/g vs. 2850 m²/g).¹⁷ The CO₂ uptake capacity of MMPF-2 at 298 K and 760 torr is 19.8 wt. % (or 101 cm³/g, or 4.51 mmol/g), which is also among the highest yet reported for porous MOFs under the same conditions.¹⁸ The isosteric heats of adsorption (Qₛₜ)for CO₂ were calculated based on the CO₂ gas adsorption isotherms at 273 K and 298 K using the virial method (FIG. 2.10).¹⁹ As shown in FIG. 2.2c, MMPF-2 exhibits a constant Qₛₜ of ˜31 kJ/mol at all loadings, distinguishing it from other MOFs with open metal sites, whose Qₛₜ usually decreases abruptly to 20-25 kJ/mol with the increase of CO₂ loading despite their high initial Qₛₜ.^{18b} We tentatively attribute this to the high density of open metal sites (˜5 open cobalt sites/nm³) in MMPF-2, since open metal sites have been well-known to contribute to interactions between CO₂ and MOF frameworks.^{18a,b}

In summary, by self-assembling the custom designed octatopic porphyrin ligand, tdcpp with the distorted cobalt trigonal prism SBU, we constructed a novel (6, 8, 8)-connected porphyrin-based MOF, MMPF-2, which features cobalt(II) metallated porphyrin macrocyles rigidly arranged in a “face-to-face” configuration. The linkage between the multitopic porphyrin ligand and the robust SBU together with the rigid cofacial arrangement of the metalloporphyrin macrocycles affords MMPF-2 b_{y} far the highest surface area of 2037 m²/g among reported porphyrin-based MOFs and interesting CO₂ capture performances. Considering the versatility of metalloporphyrins, this work lays a solid foundation for developing porphyrin-based MOFs as a type of functional materials for applications in gas storage, CO₂ capture, heterogeneous catalysis, sensing, etc.

References, each of which is incorporated herein by reference:
1 K. M. Kadish, K. M. Smith and R. Guilard and Eds., The Porphyrin Handbook, Academic Press: San Diego, 2000-2003.
2 J. S. Lindsey, In The Porphyrin Handbook; K. M. Kadish, K. M. Smith and R. Guilard, Eds.; Academic Press: San Diego, 2000; Vol. 1; pp 45-118.
3 J.-H. Chou, M. E. Kosal, H. S. Nalwa, N. A. Rakow and K. S. Suslick, In The

Porphyrin Handbook; K. M. Kadish, K. M. Smith and R. Guilard, Eds.; Academic Press: New York, 2000; Vol. 6, Chapter 41, pp 43-131.
4 a) Y. Nakamura, N. Aratani and A. Osuka, Chem. Soc. Rev., 2007, 36, 831-845; b) I. Beletskaya, V. S. Tyurin, A. Y. Tsivadze, R. Guilard and C. Stern, Chem. Rev., 2009, 109, 1659-1713; c) C. M. Drain, A. Varotto and I. Radivojevic, Chem. Rev., 2009, 109, 1630-1658.
5 a) M. E. Kosal and K. S. Suslick, J. Solid State Chem., 2000, 152, 87-98; b) M. E. Kosal, J. H. Chou, S. R. Wilson and K. S. Suslick, Nat. Mater., 2002, 1, 118-121; c) K. S. Suslick, P. Bhyrappa, J. H. Chou, M. E. Kosal, S. Nakagaki, D. W. Smithenry and S. R. Wilson, Acc. Chem. Res., 2005, 38, 283-291; d) B. J. Burnett, P. M. Barron, C. Hu and W. Choe, J. Am. Chem. Soc., 2011, 133, 9984-9987; e) L. D. DeVries, P. M. Barron, E. P. Hurley, C. Hu and W. Choe, J. Am. Chem. Soc., 2011, 133, 14848-14851; f) C. Y. Lee, 0. K. Farha, B. J. Hong, A. A. Sarjeant, S. B. T. Nguyen and J. T. Hupp, J. Am. Chem. Soc., 2011, 133, 15858-15861.
6 B. F. Abrahams, B. F. Hoskins and R. Robson, J. Am. Chem. Soc., 1991, 113, 3606-3607.
7 a) I. Goldberg, Chem. Commun., 2005, 1243-1254; b) I. Goldberg, Cryst Eng Comm, 2008, 10, 637-645.
8 a) A. M. Shultz, O. K. Farha, J. T. Hupp and S. T. Nguyen, J. Am. Chem. Soc., 2009, 131, 4204-4205; b) O. K. Farha, A. M. Shultz, A. A. Sarjeant, S. T. Nguyen and J. T. Hupp, J. Am. Chem. Soc., 2011, 133, 5652-5655.
9 a) E. Y. Choi, C. A. Wray, C. H. Hu and W. Choe, Cryst Eng Comm, 2009, 11, 553-555; b) S. Matsunaga, N. Endo, W. Mori, Eur. J. Inorg. Chem., 2011, 2011, 4550-4557.
10 a) M. H. Xie, X. L. Yang and C. D. Wu, Chem. Commun., 2011, 47, 5521-5523; b) M. H. Xie, X. L. Yang, C. Zou and C. D. Wu, Inorg Chem, 2011, 50, 5318-5320; c) C. Zou, Z. Zhang, X. Xu, Q. Gong, J. Li and C.-D. Wu, J. Am. Chem. Soc., 2011, 133, 87-90.
11 a) D. W. Smithenry, S. R. Wilson and K. S. Suslick, Inorg. Chem., 2003, 42, 7719-7721; b) T. Ohmura, A. Usuki, K. Fukumori, T. Ohta, M. Ito and K. Tatsumi, Inorg. Chem., 2006, 45, 7988-7990; c) X.-S. Wang, L. Meng, Q. Cheng, C. Kim, L. Wojtas, M. Chrzanowski, Y.-S. Chen, X. P. Zhang and S. Ma, J. Am. Chem. Soc., 2011, 133, 16322-16325; d) A. Fateeva, S. Devautour-Vinot, N. Heymans, T. Devic, J.-M. Greneche, S. Wuttke, S. Miller, A. Lago, C. Serre, W. G. De, G. Maurin, A. Vimont and G. Ferey, Chem. Mater., 2011, 23, 4641-4651; e) X.-S. Wang, M. Chrzanowski, W.-Y. Gao, L. Wojtas, Y.-S. Chen, M. J. Zaworotko and S. Ma, unpublished results.
12 RCSR (Reticular Chemistry Structure Resource), the original database is available for free at http://rcsr.anu.edu.au/.
13 J. Kim, B. Chen, T. M. Reineke, H. Li, M. Eddaoudi, D. B. Moler, M. O'Keeffe and O. M. Yaghi, J. Am. Chem. Soc., 2001, 123, 8239-8247.
14. G. Ferey, C. Mellot-Draznieks, C. Serre, F. Millange, J. Dutour, S. Surble and I. Margiolaki, Science, 2005, 309, 2040-2042.
15 V. A. Blatov, IUCr Comp. Comm. Newsletter, 2006, 7, 4.
16 A. L. Spek, J Appl Crystallogr, 2003, 36, 7-13.
17. Y.-G. Lee, H. R. Moon, Y. E. Cheon and M. P. Suh, Angew. Chem. Int. Ed., 2008, 47, 7741-7745.
18. a) J. -R. Li, Y. Ma, M. C. McCarthy, J. Sculley, J. Yu, H. -K. Jeong, P. B. Balbuena and H. -C. Zhou, Coord. Chem. Rev., 2011, 255, 1791-1823; b) K. Sumida, D. L. Rogow, J. A. Mason, T. M. McDonald, E. D. Bloch, Z. R. Herm, T. -H. Bae and J. R. Long, Chem. Rev., 2012, 112, 724-781; c) J. An, S. J. Geib, N. L. Rosi, J. Am. Chem. Soc., 2010, 132, 38-39; d) R. Vaidhyanathan, S. S. Iremonger, G. K. H. Shimizu, P. G. Boyd, S. Alavi, T. K. Woo, Science 2010, 330, 650-653; e) S. D. Burd, S. Ma, J. A. Perman, B. J. Sikora, R. Q. Snurr, P. K. Thallapally, J. Tian, L. Wojtas, M. J. Zaworotko, J. Am. Chem. Soc. 2012, 134, 3363-3366; e) B. Li, Z. Zhang, Y. Li, K. Yao, Y. Zhu, Z. Deng, F. Yang, X. Zhou, G. Li, H. Wu, N. Nijem, Y. J. Chabal, Z. Lai, Y. Han, Z. Shi, S. Feng and J. Li, Angew. Chem. Int. Ed., 2012, 51, 1412-1415; f) W.-Y. Gao, W. Yan, R. Cai, L. Meng, A. Salas, X.-S. Wang, L. Wojtas, X. Shi, and S. Ma, Inorg. Chem., 2012, 51, 4423-4425.
19 L. Czepirski, J. Jagiello, Chem. Eng. Sci. 1989, 44, 797-801.

### Supplemental Material:

### General Methods.

Commercially available reagents were purchased as high purity from Fisher Scientific or Frontier Scientific and used without further purification. Tetrakis(3,5-dicarboxyphenyl)porphine (H₁₀tdcpp) was synthesized by the literature.^{1,2} Solvents were purified according to standard methods and stored in the presence of molecular sieve. Thermogravimetric analysis (TGA) was performed under nitrogen on a TA Instrument TGA 2950 Hi-Res. (See FIGS. 2.5-2.13)

### Synthesis of MMPF-2:

A mixture of H₁₀tdcpp (0.001 g), Co(NO₃)₂.6H₂O (0.003 _{g}), and 1.0 mL mixture solvent (DMA(dimethylacetamide):MeOH:H₂O=4:1:1) was sealed in a Pyrex tube under vacuum and heated to 115° C. for 24 hours. The resulting dark red block crystals were washed with DMA to give pure MMPF-2 {[Co₃(OH)(H₂O)₄](CoHtdcpp)₃}.(H₂O)₂₀.(CH₃OH)₂₂.(C₄H₉NO)₂₅ (yield: 60% based on tdcpp). Anal. Calc. for MMPF-2: C, 47.53; H, 6.18; N, 7.38; Found: C, 48.99; H, 6.08; N, 7.58.

### Single-Crystal X-Ray Diffraction Studies of MMPF-2:

The X-ray diffraction data were collected using synchrotron radiation, λ=0.40663 Å, at Advanced Photon Source, Argonne National Laboratory. Indexing was performed using APEX2³ (Difference Vectors method). Data integration and reduction were performed using SaintPlus 6.01⁴. Absorption correction was performed by multi-scan method implemented in SADABS.5 Space groups were determined using XPREP implemented in APEX2.³ The structure was solved using SHELXS-97 (direct methods) and refined using SHELXL-97 (full-matrix least-squares on F2) contained in APEX2³ and WinGX v1.70.01⁶⁻⁹ programs packages. Despite of using synchrotron source and trying several crystals from different batches, diffraction experiment resulted in low quality diffraction data (lack of high angle reflections). This can be attributed to the presence of the ligand/solvent disorder and to the presence of bad quality, multiply twinned crystals. Due to the low resolution of the data, C, N, O atoms were refined with isotropic displacement parameters and disordered ligand moiety was refined using distance restraints. Hydrogen atoms were placed in geometrically calculated positions and included in the refinement process using riding model with isotropic thermal parameters: Uiso(H)=1.2Ueq(-CH). The contribution of disordered solvent molecules was treated as diffuse using Squeeze procedure implemented in Platon program.^{10,11} Crystal data and refinement conditions are shown in Table S2. The framework is neutral: μ-OH— is located in the center of Co-trimer and the negative charge is balanced by H+ cations, located between O3 . . . O3′ carboxylate oxygen atoms. Crystal data and refinement conditions are shown in Table S2. Crystallographic data have been deposited with the Cambridge Crystallographic Data Centre: CCDC 840130, this data can be obtained free of charge from The Cambridge Crystallographic Data Center via www.ccdc.cam.ac.uk/data request/cif.

The MMPF-2 structure has been solved and refined in P4/mbm space group. There are six porphyrin moieties and 30 Co cations in the unit cell. There are two independent porphyrin moieties in the structure with Co1 and Co4 core metals. Both porphyrin moieties are located on symmetry elements so that Co1 atom is located at a site with mmm symmetry (d Wyckoff position) and Co4 is located at site with m.2m symmetry (g Wyckoff position). Consequently there is ⅛ of Co1-porphyrin moiety and ¼ of Co4-porphyrin moiety in the asymmetric unit. N1 and N2 nitrogen atoms of Co1-porphyrin are located on 2-fold axis and two mirror planes (2.mm and m.2m site symmetries respectively) while N3 and N4 nitrogen atoms of Co4-porphyrin are located on a mirror plane ( . . . m and m . . . site symmetries respectively).

### Gas Adsorption Experiments.

Gas adsorption isotherms of MMPF-2 were collected using the surface area analyzer ASAP-2020. Before the measurements, the freshly prepared samples were soaked with methanol, and then were activated using the Supercritical CO₂ Dryer according to the procedures reported in the literature.¹² N₂, Ar, and O₂ gas adsorption isotherms were measured at 77 K or 87K using a liquid N₂ or Ar bath, respectively, and CO₂ gas adsorption isotherms were measured at 273 K and 298 K using an ice-water bath and 298 K water bath respectively.

### Isosteric Heat of Adsorption (Qst) Calculations.

The virial equation of the form given in Equation (1)¹³ was employed to calculate the enthalpies of adsorption for CO2 on MMPF-2.

ln P=lnN+1/T Σᵢ₌₀^{m}aᵢNⁱ+Σᵢ₌₀ⁿbᵢNⁱ (1)

where P is the pressure expressed in Torr, N is the amount adsorbed in mmol/g, T is the temperature in K, aᵢ and bᵢ are virial coefficients, and m and n represent the number of coefficients required to adequately describe the isotherms. The equation was fitted by using the the least-squares method; m and n were gradually increased until the contribution of a and b coefficients toward the overall fitting is statistically trivial, as determined by the t-test. The values of the virial coefficients a0 . . . am were then used to calculate the isosteric heat of adsorption by the following expression:

Qₛₜ=−R Σᵢ₌₀^{m}aᵢNⁱ (2)

References, each of which is incorporated herein by reference:
1. P. Bhyrappa, G. Vaijayanthimala, B. Verghese, Tetrahedron Letters 2002, 43, 6427-6429.
2. W. Fudickar, J. Zimmermann, L. Ruhlmann, J. Schneider, B. Roeder, U. Siggel, J. -H. Fuhrhop, J. Am. Chem. Soc. 1999, 121, 9539-9545.
3. Bruker, 2010, APEX2. Bruker AXS Inc., Madison, Wis., USA.
4. Bruker, 2009, SAINT. Data Reduction Software. Bruker AXS Inc., Madison, Wis., USA.
5. G. M. Sheldrick, 2008, SADABS. Program for Empirical Absorption. Correction. University of Gottingen, Germany.
6. L. Farrugia, J App. Cryst. 1999, 32, 837.
7. G. M. Sheldrick, 1997, SHELXL-97. Program for the Refinement of Crystal.
8. G. M. Sheldrick, Acta Cryst. 1990, A46, 467.
9. G. M. Sheldrick, Acta Cryst. 2008, A64, 112.
10. T. L. Spek, Acta Cryst. 1990, A46, 194-201.
11. T. L. Spek, Acta Cryst., 1990, A46, c34.
12. A. P. Nelson, O. K. Farha, K. L. Mulfort, J. T. Hupp, J. Am. Chem. Soc. 2009, 131, 458-460.
13. L. Czepirski, J. Jagiello, Chem. Eng. Sci. 1989, 44, 797-801.
14. A. L. Myers, J. M. Prausnitz, AlChE J. 1965, 11, 121-127.
15. B. F. Abrahams, B. F. Hoskins, R. Robson, J. Am. Chem. Soc. 1991, 113, 3606-3607.
16. B. F. Abrahams, B. F. Hoskins, D. M. Michail, R. Robson, Nature 1994, 369, 727-729.
17. D. Hagrman, P. J. Hagrman, J. Zubieta, Angew. Chem. Int. Ed. 1999, 38, 3165-3168.
18. K. J. Lin, Angew. Chem. Int. Ed. 1999, 38, 2730-2732.
19. C. V. K. Sharma, G. A. Broker, J. G. Huddleston, J. W. Baldwin, R. M. Metzger, R. D. Rogers, J. Am. Chem. Soc. 1999, 121, 1137-1144.
20. Y. Diskin-Posner, G. K. Patra, I. Goldberg, Dalton Trans. 2001, 2775-2782
21. M. E. Kosal, J. H. Chou, S. R. Wilson, K. S. Suslick, Nat. Mater. 2002, 1, 118-121
22. L. Pan, S. Kelly, X. Y. Huang, J. Li, Chem. Commun. 2002, 2334-2335.
23. D. Sun, F. S. Tham, C. A. Reed, P. D. W. Boyd, Proc. Natl. Acad. Sci. USA 2002, 99, 5088-5092.
24. B. Zimmer, V. Bulach, M. W. Hosseini, A. De Cian, N. Kyritsakas, Eur. J. Inorg. Chem. 2002, 3079-3082.
25. L. Carlucci, G. Ciani, D. M. Proserpio, F. Porta, Angew. Chem. Int. Ed. 2003, 42, 317-322.
26. D. W. Smithenry, S. R. Wilson, K. S. Suslick, Inorg. Chem. 2003, 42, 7719-7721.
27. M. Shmilovits, M. Vinodu, I. Goldberg, Cryst. Growth Des. 2004, 4, 633-638.
28. M. Shmilovits, M. Vinodu, I. Goldberg, New J. Chem. 2004, 28, 223-227.
29. G. Yucesan, V. Golub, C. J. O'Connor, J. Zubieta, Cryst Eng Comm 2004, 6, 323-325.
30. L. Carlucci, G. Ciani, D. M. Proserpio, F. Porta, Cryst Eng Comm 2005, 7, 78-86.
31. E. Deiters, V. Bulach, M. W. Hosseini, Chem. Commun. 2005, 3906-3908.
32. S. K. Taylor, G. B. Jameson, P. D. W. Boyd, Supramol. Chem. 2005, 17, 543-546.
33. K. S. Suslick, P. Bhyrappa, J. H. Chou, M. E. Kosal, S. Nakagaki, D. W. Smithenry, S. R. Wilson, Accounts Chem. Res. 2005, 38, 283-291.
34. R. Kempe, Z. Anorg. Allg. Chem. 2005, 631, 1038-1040.
35. S. George, S. Lipstman, I. Goldberg, Cryst. Growth Des. 2006, 6, 2651-2654.
36. T. Ohmura, A. Usuki, K. Fukumori, T. Ohta, M. Ito, K. Tatsumi, Inorg. Chem. 2006, 45, 7988-7990.
37. N. Zheng, J. Zhang, X. Bu, P. Feng, Cryst. Growth Des. 2007, 7, 2576-2581.
38. E. Y. Choi, P. M. Barron, R. W. Novotney, C. H. Hu, Y. U. Kwon, W. Y. Choe, Cryst Eng Comm 2008, 10, 824-826.
39. S. Lipstman, S. Muniappan, I. Goldberg, Cryst. Growth Des. 2008, 8, 1682-1688.
40. E. Kuhn, V. Bulach, M. W. Hosseini, Chem. Commun. 2008, 5104-5106.
41. S. Lipstman, I. Goldberg, J. Mol. Struct. 2008, 890, 101-106.
42. P. M. Barron, H. T. Son, C. H. Hu, W. Choe, Cryst. Growth Des. 2009, 9, 1960-1965.
43.W. T. Chen, S. Fukuzumi, Eur. J. Inorg. Chem. 2009, 5494-5505.
44. E. Y. Choi, P. M. Barron, R. W. Novotny, H. T. Son, C. Hu, W. Choe, Inorg. Chem. 2009, 48, 426-428.
45. E. Y. Choi, C. A. Wray, C. H. Hu, W. Choe, Cryst Eng Comm 2009, 11, 553-555.
46. H. Chung, P. M. Barron, R. W. Novotny, H. T. Son, C. Hu, W. Choe, Cryst. Growth Des. 2009, 9, 3327-3332.
47. A. M. Shultz, O. K. Farha, J. T. Hupp, S. T. Nguyen, J. Am. Chem. Soc. 2009, 131, 4204-4205.
48. R. W. Seidel, I. M. Oppel, Struct. Chem. 2009, 20, 121-128.
49. J. M. Verduzco, H. Chung, C. H. Hu, W. Choe, Inorg. Chem. 2009, 48, 9060-9062.
50. P. M. Barron, C. A. Wray, C. Hu, Z. Guo, W. Choe. Inorg. Chem. 2010, 49, 10217-10219.
51. E. Y. Choi, L. D. DeVries, R. W. Novotny, C. H. Hu, W. Choe, Cryst. Growth Des. 2010, 10, 171-176.
52. S. Lipstman, I. Goldberg, Cryst Eng Comm 2010, 12, 52-54.
53. R. W. Seidel, I. M. Oppel, Cryst Eng Comm 2010, 12, 1051-1053.
54. R. W. Seidel, I. M. Oppel, Z. Anorg. Allg. Chem. 2010, 636, 446-448.
55. B. J. Burnett, P. M. Barron, C. Hu, W. Choe, J. Am. Chem. Soc. 2011, 133, 9984-9987.
56. L. D. DeVries, P. M. Barron, E. P. Hurley, C. Hu, W. Choe, J. Am. Chem. Soc. 2011, 133, 14848-14851.
57. X. -S. Wang, L. Meng, Q. Cheng, C. Kim, L. Wojtas, M. Chrzanowski, Y. -S. Chen, X. P. Zhang, S. Ma, J. Am. Chem. Soc. 2011, 133, 16322-16325.
58. M. H. Xie, X. L. Yang, C. D. Wu, Chem. Commun. 2011, 47, 5521-5523.
59. M. H. Xie, X. L. Yang, C. Zou, C. D. Wu, Inorg. Chem. 2011, 50, 5318-532.
60. O. K. Farha, A. M. Shultz, A. A. Sarjeant, S. T. Nguyen, J. T. Hupp, J. Am. Chem. Soc. 2011, 133, 5652-5655.
61. C. Y. Lee, O. K. Farha, B. J. Hong, A. A. Sarjeant, S. B. T. Nguyen and J. T. Hupp, J. Am. Chem. Soc., 2011, 133, 15858-15861.
62. A. Fateeva, S. Devautour-Vinot, N. Heymans, T. Devic, J. -M. Greneche, S. Wuttke, S. Miller, A. Lago, C. Serre, W. G. De, G. Maurin, A. Vimont, G. Ferey, Chem. Mater. 2011, 23, 4641-4651.
63. S. Matsunaga, N. Endo, W. Mori, Eur. J. Inorg. Chem. 2011, 4550-4557.
64. C. Zou, Z. Zhang, X. Xu, Q. Gong, J. Li, C.-D. Wu, J. Am. Chem. Soc., 2012, 134, 87-90.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to measurement technique and/or the numerical value. In addition, the phrase “about ‘x’ to ‘y’” includes “about to about ‘y’”.

Many variations and modifications may be made to the above-described embodiments. All such modifications and variations are intended to be included herein within the scope of this disclosure and protected by the following claims.

We claim:

1. A composition comprising:
a metal-metalloporphyrin framework that includes a porphyrin ligand and a secondary building unit, wherein the porphyrin ligand is represented by formula A: 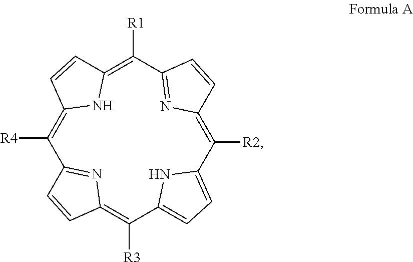
wherein one or more of the R1, R2, R3, and R4, includes a functional group that bonds with the secondary building unit, wherein R1, R2, R3, and R4 are independently selected from H and a moiety having one of more functional groups selected from the group consisting of: —CO₂H, —CS₂H, —B(OH)₂, —SO₃H, —CN, -tetrazolate, -1,2,3 or 1,2,4-triazolate, -pyrazolate, —PO₃H, and -pyridyl; wherein at least one of R1, R2, R3, and R4 is not H.

2. The composition of claim 1, wherein the R1, R2, R3, and R4 are independently selected from: H, a polycarboxylated ligand, a polypyridyl ligand, a polycyano ligand, a polyphosphonate ligand, a polyhydroxyl ligand, a polysulfonate ligand, a polyimidazolate, ligand, a polytriazolate ligand, and a combination thereof, wherein at least one of R1, R2, R3, and R4 is not H.

3. The composition of claim 1, wherein the two of R1, R2, R3, and R4 are H and the other two are independently selected the moiety.

4. The composition of claim 3, wherein the moiety is an aromatic dicarboxylic acid moiety.

5. The composition of claim 1, wherein R1 and R3 are H and R2 and R4 are an isophthalic acid moiety.

6. The composition of claim 1, wherein each of R1, R2, R3, and R4 are independently selected the moiety.

7. The composition of claim 6, wherein the moiety is an aromatic dicarboxylic acid moiety.

8. The composition of claim 6, wherein the moiety is an isophthalic acid moiety.

9. The composition of claim 1, wherein the secondary building unit includes a metal.

10. The composition of claim 1, wherein the secondary building unit is selected from the group consisting of: a dicopper paddlewheel secondary building unit, a distorted dicobalt trigonal prism secondary building unit, a dimetal secondary building unit, a square paddlewheel secondary building unit, a dimetal triangular paddlewheel secondary building unit, a tetra-metal clusters secondary building unit, and a single metal ion secondary building unit.

11. The composition of claim 1, further comprising a plurality of porphyrin ligands and secondary building units that are connected to one another to form one or more nanoscopic cages.

12. The composition of claim 11, wherein a nanoscopic cage includes eight dicopper paddlewheel secondary building units and sixteen porphyrin ligands, wherein R1 and R3 are H and R2 and R4 are an isophthalic acid moiety.

13. The composition of claim 11, wherein a nanoscopic cage includes a plurality of dicobalt trigonal prism secondary building units and plurality of porphyrin ligands, wherein each of R1, R2, R3, and R4 are an isophthalic acid moiety.

14. A metal-organic polyhedron (MOP), comprising:
a porphyrin ligand and a secondary building unit, wherein the one or more of the R1, R2, R3, and R4, include a functional group that bonds with the secondary building unit; wherein the porphyrin ligand is represented by formula A: 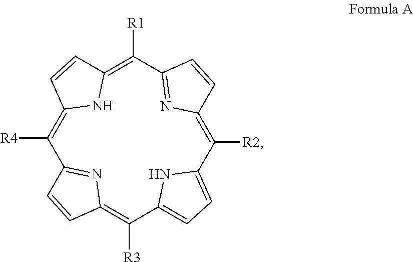
wherein R1, R2, R3, and R4 are independently selected from H and a moiety having one of more functional groups selected from the group consisting of: —CO₂H, —CS₂H, —NO₂, —B(OH)₂, —SO₃H, —CN, -tetrazolate, -1,2,3 or 1,2,4-triazolate, -pyrazolate, —PO₃H, and -pyridyl; wherein at least one of R1, R2, R3, and R4 is not H.

15. The MOP of claim 14, wherein R1 and R3 are H and R2 and R4 are an isophthalic acid moiety.

16. The MOP of claim 14, wherein each of R1, R2, R3, and R4 are an isophthalic acid moiety.

17. The MOP of claim 14, wherein the MOP includes eight dicopper paddlewheel secondary building units and sixteen porphyrin ligands, wherein R1 and R3 are H and R2 and R4 are an isophthalic acid moiety.

18. The MOP of claim 14, wherein the MOP includes an secondary building unit selected from the group consisting of: a dimetal secondary building unit, a square paddlewheel secondary building unit, a dimetal triangular paddlewheel secondary building unit, a tetra-metal clusters secondary building unit, a single metal ion secondary building unit, and a dicobalt trigonal prism secondary building unit; and a porphyrin ligand, wherein each of R1, R2, R3, and R4 are an isophthalic acid moiety
